(19) United States
(12) Patent Application Publication
(10) Pub. No.: US 2017/0152490 A9
(43) Pub. Date: Jun. 1, 2017

(54) USE OF ACTIVIN RECEPTOR-LIKE KINASE 1 (ALK-1) ANTAGONISTS IN THE TREATMENT OF CANCER

(71) Applicant: Acceleron Pharma, Inc., Cambridge, MA (US)

(72) Inventors: Marat B. Alimzhanov, Wellesley, MA (US); Shuchi Sumant Pandya, Newton, MA (US)

(73) Assignee: Acceleron Pharma, Inc., Cambridge, MA (US)

(21) Appl. No.: 14/671,064

(22) Filed: Mar. 27, 2015

Related U.S. Application Data
(60) Provisional application No. 61/972,204, filed on Mar. 28, 2014.

(51) Int. Cl. C12N 9/12; A61K 38/45; A61K 39/395; C12Q 1/70; A61K 33/24

(52) U.S. Cl. CPC C12N 9/12; C12Q 1/708; A61K 33/24; A61K 39/3955; A61K 38/45; C12Q 2600/136; C12Y 207/1103; C07K 2319/30

(57) ABSTRACT

Some aspects of this disclosure provide methods and compositions for the treatment of cancer, for example, head and neck cancer, in a subject using ALK1 inhibitors, e.g., ALK1-ECD polypeptides, ALK1-Fc fusion proteins, or ALK1-inhibitory antibodies. In some embodiments, methods and compositions are provided for treating certain cancers with ALK1 inhibitors in combination with a chemotherapeutic platinum agent. In some embodiments, methods are provided for identifying whether a cancer in a subject will react to treatment with an ALK1 antagonist, either alone or in combination with a chemotherapeutic platinum agent, for example, based on a determination that the cancer or the subject is positive for human papilloma virus.

### RELATED APPLICATIONS

This application claims the benefit of the filing date under 35 U.S.C. §119(e) to United States Provisional Patent Application Serial Number U.S. 61/972,204, filed Mar. 28, 2014, entitled “Use of Activin Receptor-Like Kinase 1 (Alk-1) Antagonists in the Treatment of Cancer,” the entire contents of which are incorporated herein by reference.

### BACKGROUND

Cancer is a leading cause of death and is characterized by the uncontrolled growth and spread of abnormal cells. Many types of tumors depend on the growth of new blood vessels (angiogenesis) to supply adequate nutrients and oxygen, allowing cancer cells to grow, invade nearby tissue, and spread to other parts of the body. Angiogenesis inhibition is a widely-used approach to cancer treatment. Several angiogenesis inhibitors that work by blocking the vascular endothelial growth factor (VEGF) pathway are approved or are in development. These therapies, given alone or in combination with chemotherapy and/or radiation, can significantly improve survival.

For example, Avastin™ (bevacizumab), a monoclonal antibody that binds to Vascular Endothelial Growth Factor (VEGF), has proven to be effective in the treatment of a variety of cancers. Macugen™, an aptamer that binds to VEGF has proven to be effective in the treatment of neovascular (wet) age-related macular degeneration. Antagonists of the SDF/CXCR4 signaling pathway inhibit tumor neovascularization and are effective against cancer in mouse models (Guleng et al. Cancer Res. 2005 July 1;65(13):5864-71). The isocoumarin 2-(8-hydroxy-6-methoxy-1-oxo-1 H-2-benzopyran-3-yl) propionic acid (NM-3) directly kills both endothelial and tumor cells in vitro and is effective in the treatment of diverse human tumor xenografts in mice (Agata et al. Cancer Chemother Pharmacol. 2005 December;56(6):610-4.). Thalidomide and related compounds have shown beneficial effects in the treatment of cancer, and the inhibition of angiogenesis appears to be an important component of its anti-tumor effect (see, e.g., Dredge et al. Microvasc Res. 2005 January;69(1-2):56-63). The success of TNF-alpha antagonists in the treatment of rheumatoid arthritis is partially attributed to anti-angiogenic effects on the inflamed joint tissue (Feldmann et al. Annu Rev Immunol. 2001;19:163-96).

While VEGF inhibitors play an important role in the treatment of certain tumors, some tumors do not react at all or react only temporarily to treatment with VEGF inhibitors alone. Accordingly, it is desirable to have additional compositions and methods for inhibiting angiogenesis in the context of cancer therapy.

### SUMMARY

Some aspects of this disclosure provide methods and compositions for the treatment of cancer in a subject. In some embodiments, methods and compositions are provided for treating certain cancers with ALK1 antagonists. In some embodiments, methods are provided for identifying whether a cancer will react to treatment with an ALK1 antagonist. Additional methods and compositions are provided for combination therapy of cancer with an ALK1 antagonist and a chemotherapeutic platinum agent.

Some aspects of this disclosure provide methods for treating head and neck cancer in a subject. In some embodiments, the method comprises administering to a subject in need thereof (a) an agent selected from the group consisting of (i) an ALK1-extracellular domain (ALK1-ECD) polypeptide; (ii) an antibody that binds to an ALK1 polypeptide comprising amino acids 22-118 of SEQ ID NO: 1; or (iii) an antibody that binds to BMP9 or BMP10; and (b) a chemotherapeutic platinum agent; in an amount sufficient to treat the head and neck cancer in the subject. In some embodiments, the amino acid sequence of the ALK1-ECD polypeptide is at least 90% identical to the sequence of amino acids 22-118 of SEQ ID NO: 1. In some embodiments, the amino acid sequence of the ALK1-ECD polypeptide is at least 97% identical to the sequence of amino acids 22-118 of SEQ ID NO: 1. In some embodiments, the C-terminal amino acid residue of the ALK1-ECD polypeptide is not glutamine 118 (Q118) of SEQ ID NO: 1. In some embodiments, the C-terminal amino acid residue of the ALK1-ECD polypeptide is not a glutamine residue. In some embodiments, the C-terminal amino acid residue of the ALK1-ECD polypeptide is proline 113 (P113), glycine 114 (G114), threonine 115 (T115), aspartic acid 116 (D116), glycine 117 (G117), leucine 119 (L119 (L119), alanine 120 (A120), leucine 121 (L121), isoleucine 122 (I122), or leucine 123 (L123) of SEQ ID NO: 1.

In some embodiments, the ALK1-ECD polypeptide is fused to an Fc portion of an immunoglobulin thus forming an ALK1-Fc fusion protein. In some embodiments, the Fc portion is an Fc portion of a human IgG1. In some embodiments, the amino acid sequence of the ALK1-Fc fusion protein comprises the sequence of SEQ ID NO: 3 or SEQ ID NO: 4. In some embodiments, the amino acid sequence of the ALK1-Fc fusion protein is at least 90% identical to the amino acid sequence of SEQ ID NO: 3 or SEQ ID NO: 4. In some embodiments, the amino acid sequence of the ALK1-Fc fusion protein is at least 97% identical to the amino acid sequence of SEQ ID NO: 3 or SEQ ID NO: 4. In some embodiments, at least 90% of the ALK1-Fc fusion protein is in a dimeric form. In some embodiments, the ALK1-ECD polypeptide and/or the ALK1-Fc fusion protein is glycosylated. In some embodiments, the ALK1-ECD polypeptide and/or the ALK1-Fc fusion protein binds BMP9 or BMP10 with a K_{D} of less than 1×10⁻⁷M, e.g., with a K_{D} of less than 1×10⁻⁸M, less than 1×10⁻⁹M, less than 1×10⁻¹⁰M, or less than 1×10⁻¹¹M. In some embodiments, the ALK1-ECD polypeptide and/or the ALK1-Fc fusion protein binds to TGFβ-1 with a K_{D} of greater than 1×10⁻⁶M. In some embodiments, the ALK1-ECD polypeptide and/or the ALK1-Fc fusion protein is administered in a pharmaceutical preparation.

In some embodiments, the antibody of (ii) binds to an ALK1-ECD polypeptide with a K_{D} of less than 5×10⁻⁸ M. In some embodiments, the antibody of (ii) binds to an ALK1-ECD polypeptide with a K_{D} of less than 1×10⁻¹⁰ M. In some embodiments, the antibody of (ii) inhibits angiogenesis stimulated by an ALK1 ligand. In some embodiments, the antibody of (ii) inhibits the binding of BMP9 or BMP10 to an ALK1-ECD polypeptide. In some embodiments, the antibody of (iii) binds to an ALK1-ECD polypeptide with a K_{D} of less than 5×10⁻⁸ M. In some embodiments, the antibody of (iii) inhibits angiogenesis stimulated by at least one ALK1 ligand.

In some embodiments, the chemotherapeutic platinum agent comprises a coordination complex of platinum. In some embodiments, the chemotherapeutic platinum agent is cisplatin, carboplatin, oxaliplatin, satraplatin, picoplatin, Nedaplatin, Triplatin, Lipoplatin, or any combination thereof.

In some embodiments, the ALK1-ECD polypeptide and/or the ALK1-Fc fusion protein is administered to the subject at a dosage of 0.1-30 mg/kg/day. In some embodiments, higher dosages are envisioned. In some embodiments, the ALK1-ECD polypeptide and/or the ALK1-Fc fusion protein is administered to the subject at a dosage of 0.1, 0.2, 0.3, 0.4, 0.5, 0.6, 0.7, 0.8, 0.9, 1, 2, 3, 4, 5, 10, 15, 20, 25, or 30 mg/kg/day. In some embodiments, the chemotherapeutic platinum agent is administered to the subject at a dosage of 0.1-10 mg/kg/day. In some embodiments, the chemotherapeutic platinum agent is administered to the subject at a dosage of 0.1, 0.2, 0.3, 0.4, 0.5, 0.6, 0.7, 0.8, 0.9, 1, 1.5, 2, 2.5, 3, 3.5, 4, 4.5, 5, 6, 7, 7.5, 8, 9, or 10 mg/kg/day. In some embodiments, the ALK1-ECD polypeptide and/or the ALK1-Fc fusion protein is administered to the subject at a dose of 10 mg/kg/day, and the chemotherapeutic platinum agent is administered to the subject at a dose of 5 mg/kg/day.

In some embodiments, the head and neck cancer is positive for human papilloma virus (HPV). In some embodiments, the method further comprises determining whether the head and neck cancer is positive for HPV. In some embodiments, the agent of (a) and the chemotherapeutic agent of (b) are administered based on the head and neck cancer being positive for HPV. In some embodiments, the head and neck cancer is a recurrent or metastatic squamous cell carcinoma.

Some aspects of this disclosure provide methods for treating HPV-positive head and neck cancer in a subject. In some embodiments, the method comprises administering to a subject in need thereof an agent selected from the group consisting of (i) an ALK1-extracellular domain (ALK1-ECD) polypeptide; (ii) an antibody that binds to an ALK1 polypeptide comprising amino acids 22-118 of SEQ ID NO: 1; and (iii) an antibody that binds to BMP9 or BMP10; in an amount sufficient to treat the head and neck cancer in the subject. In some embodiments, the amino acid sequence of the ALK1-ECD polypeptide is at least 90% identical to the sequence of amino acids 22-118 of SEQ ID NO: 1. In some embodiments, the amino acid sequence of the ALK1-ECD polypeptide is at least 97% identical to the sequence of amino acids 22-118 of SEQ ID NO: 1. In some embodiments, the C-terminal amino acid residue of the ALK1-ECD polypeptide is not glutamine 118 (Q118) of SEQ ID NO: 1. In some embodiments, the C-terminal amino acid residue of the ALK1-ECD polypeptide is not a glutamine residue. In some embodiments, the C-terminal amino acid residue of the ALK1-ECD polypeptide is proline 113 (P113), glycine 114 (G114), threonine 115 (T115), aspartic acid 116 (D116), glycine 117 (G117), leucine 119 (L119), alanine 120 (Al20), leucine 121 (L121), isoleucine 122 (I122), or leucine 123 (L123) of SEQ ID NO: 1.

In some embodiments, the ALK1-ECD polypeptide is fused to an Fc portion of an immunoglobulin thus forming an ALK1-Fc fusion protein. In some embodiments, the Fc portion is an Fc portion of a human IgG1. In some embodiments, the amino acid sequence of the ALK1-Fc fusion protein comprises the sequence of SEQ ID NO: 3 or SEQ ID NO: 4. In some embodiments, the amino acid sequence of the ALK1-Fc fusion protein is at least 90% identical to the amino acid sequence of SEQ ID NO: 3 or SEQ ID NO: 4. In some embodiments, the amino acid sequence of the ALK1-Fc fusion protein is at least 97% identical to the amino acid sequence of SEQ ID NO: 3 or SEQ ID NO: 4 In some embodiments, at least 90% of the ALK1-Fc fusion protein is in a dimeric form. In some embodiments, the ALK1-ECD polypeptide and/or the ALK1-Fc fusion protein is glycosylated. In some embodiments, the ALK1-ECD polypeptide and/or the ALK1-Fc fusion protein binds BMP9 or BMP10 with a K_{D} of less than 1×10⁻⁷M, e.g., with a K_{D} of less than 1×10⁻⁸M, less than 1×10⁻⁹M, less than 1×10⁻¹⁰M, or less than 1×10⁻¹¹M. In some embodiments, the ALK1-ECD polypeptide and/or the ALK1-Fc fusion protein binds to TGFβ3-1 with a K_{D} of greater than 1×10⁻⁶M. In some embodiments, the ALK1-ECD polypeptide and/or the ALK1-Fc fusion protein is administered in a pharmaceutical preparation.

In some embodiments, the antibody of (ii) binds to an ALK1-ECD polypeptide with a K_{D} of less than 5×10⁻⁸ M. In some embodiments, the antibody of (ii) binds to an ALK1-ECD polypeptide with a K_{D} of less than 1×10⁻¹⁰ M. In some embodiments, the antibody of (ii) inhibits angiogenesis stimulated by an ALK1 ligand. In some embodiments, the antibody of (ii) inhibits the binding of BMP9 or BMP10 to an ALK1-ECD polypeptide. In some embodiments, the antibody of (iii) binds to an ALK1-ECD polypeptide with a K_{D} of less than 5×10⁻⁸ M. In some embodiments, the antibody of (iii) inhibits angiogenesis stimulated by at least one ALK1 ligand.

In some embodiments, the ALK1-ECD polypeptide and/or the ALK1-Fc fusion protein is administered to the subject at a dosage of 0.1-30 mg/kg/day. In some embodiments, the ALK1-ECD polypeptide and/or the ALK1-Fc fusion protein is administered to the subject at a dosage of 0.1, 0.2, 0.3, 0.4, 0.5, 0.6, 0.7, 0.8, 0.9, 1, 2, 3, 4, 5, 10, 15, 20, 25, or 30 mg/kg/day.

In some embodiments, the head and neck cancer is a recurrent or metastatic squamous cell carcinoma. In some embodiments, the method further comprises determining that the head and neck cancer is HPV-positive. In some embodiments, the method further comprises identifying the head and neck cancer as responsive to treatment with the agent based on the head and neck cancer being positive for HPV. In some embodiments, the agent are administered based on the head and neck cancer being positive for HPV.

In some embodiments, the method further comprises administering a chemotherapeutic platinum agent to the subject. In some embodiments, the chemotherapeutic platinum agent comprises a coordination complex of platinum. In some embodiments, the chemotherapeutic platinum agent is cisplatin, carboplatin, oxaliplatin, satraplatin, picoplatin, Nedaplatin, Triplatin, Lipoplatin, or any combination thereof.

In some embodiments, the chemotherapeutic platinum agent is administered to the subject at a dosage of 0.1-10 mg/kg/day. In some embodiments, the chemotherapeutic platinum agent is administered to the subject at a dosage of 0.1, 0.2, 0.3, 0.4, 0.5, 0.6, 0.7, 0.8, 0.9, 1, 1.5, 2, 2.5, 3, 3.5, 4, 4.5, 5, 6, 7, 7.5, 8, 9, or 10 mg/kg/day. In some embodiments, the ALK1-ECD polypeptide and/or the ALK1-Fc fusion protein is administered to the subject at a dose of 10 mg/kg/day, and the chemotherapeutic platinum agent is administered to the subject at a dose of 5 mg/kg/day.

Some aspects of this disclosure provide methods for identifying whether a cancer responds to treatment with an ALK1 antagonist. In some embodiments, the method comprises (a) determining whether a head and neck cancer in a subject is HPV positive; wherein, if the head and neck cancer is positive for HPV, then the cancer is identified to respond to treatment with an ALK1 antagonist. In some embodiments, the ALK1 antagonist is an agent selected from the group consisting of (i) an ALK1-extracellular domain (ALK1-ECD) polypeptide; (ii) an antibody that binds to an ALK1 polypeptide comprising amino acids 22-118 of SEQ ID NO: 1; and (iii) an antibody that binds to BMP9 or BMP10. In some embodiments, the method further comprises (b) administering the ALK1 antagonist to the subject in an amount effective to treat the head and neck cancer. In some embodiments, the method further comprises obtaining a biopsy from the head and neck cancer in the subject.

In some embodiments, the amino acid sequence of the ALK1-ECD polypeptide is at least 90% identical to the sequence of amino acids 22-118 of SEQ ID NO: 1. In some embodiments, the amino acid sequence of the ALK1-ECD polypeptide is at least 97% identical to the sequence of amino acids 22-118 of SEQ ID NO: 1. In some embodiments, the C-terminal amino acid residue of the ALK1-ECD polypeptide is not glutamine 118 (Q118) of SEQ ID NO: 1. In some embodiments, the C-terminal amino acid residue of the ALK1-ECD polypeptide is not a glutamine residue. In some embodiments, the C-terminal amino acid residue of the ALK1-ECD polypeptide is proline 113 (P113), glycine 114 (G114), threonine 115 (T115), aspartic acid 116 (D116), glycine 117 (G117), leucine 119 (L119), alanine 120 (A120), leucine 121 (L121), isoleucine 122 (I122), or leucine 123 (L123) of SEQ ID NO: 1. In some embodiments, the ALK1-ECD polypeptide is fused to an Fc portion of an immunoglobulin thus forming an ALK1-Fc fusion protein. In some embodiments, the Fc portion is an Fc portion of a human IgG1. In some embodiments, the amino acid sequence of the ALK1-Fc fusion protein comprises the sequence of SEQ ID NO: 3 OR SEQ ID NO: 4. In some embodiments, the amino acid sequence of the ALK1-Fc fusion protein is at least 90% identical to the amino acid sequence of SEQ ID NO: 3 or SEQ ID NO: 4. In some embodiments, the amino acid sequence of the ALK1-Fc fusion protein is at least 97% identical to the amino acid sequence of SEQ ID NO: 3 or SEQ ID NO: 4. In some embodiments, at least 90% of the ALK1-Fc fusion protein is in a dimeric form. In some embodiments, the ALK1-ECD polypeptide and/or the ALK1-Fc fusion protein is glycosylated. In some embodiments, the ALK1-Fc fusion protein is Dalantercept. In some embodiments, the ALK1-ECD polypeptide and/or the ALK1-Fc fusion protein is administered to the subject at a dosage of 0.1-30 mg/kg/day. In some embodiments, the head and neck cancer is a recurrent or metastatic squamous cell carcinoma.

Some aspects of this disclosure provide methods that comprise (a) obtaining a biopsy from a head and neck cancer in a subject; (b) determining whether the head and neck cancer is HPV positive; and (c) if the head and neck cancer is positive for HPV, identifying the head and neck cancer as responsive to treatment with an agent selected from the group consisting of (i) an ALK1-extracellular domain (ALK1-ECD) polypeptide; (ii) an antibody that binds to an ALK1 polypeptide comprising amino acids 22-118 of SEQ ID NO: 1; and (iii) an antibody that binds to BMP9 or BMP10. In some embodiments, the method further comprises administering the agent to the subject in an amount effective to treat the head and neck cancer.

In some embodiments, the amino acid sequence of the ALK1-ECD polypeptide is at least 90% identical to the sequence of amino acids 22-118 of SEQ ID NO: 1. In some embodiments, the amino acid sequence of the ALK1-ECD polypeptide is at least 97% identical to the sequence of amino acids 22-118 of SEQ ID NO: 1. In some embodiments, the C-terminal amino acid residue of the ALK1-ECD polypeptide is not glutamine 118 (Q118) of SEQ ID NO: 1. In some embodiments, the C-terminal amino acid residue of the ALK1-ECD polypeptide is not a glutamine residue. In some embodiments, the C-terminal amino acid residue of the ALK1-ECD polypeptide is proline 113 (P113), glycine 114 (G114), threonine 115 (T115), aspartic acid 116 (D116), glycine 117 (G117), leucine 119 (L119), alanine 120 (A120), leucine 121 (L121), isoleucine 122 (I122), or leucine 123 (L123) of SEQ ID NO: 1. In some embodiments, the ALK1-ECD polypeptide is fused to an Fc portion of an immunoglobulin thus forming an ALK1-Fc fusion protein. In some embodiments, the Fc portion is an Fc portion of a human IgG1. In some embodiments, the amino acid sequence of the ALK1-Fc fusion protein comprises the sequence of SEQ ID NO: 3 or SEQ ID NO: 4. In some embodiments, the amino acid sequence of the ALK1-Fc fusion protein is at least 90% identical to the amino acid sequence of SEQ ID NO: 3 or SEQ ID NO: 4. In some embodiments, the amino acid sequence of the ALK1-Fc fusion protein is at least 97% identical to the amino acid sequence of SEQ ID NO: 3 or SEQ ID NO: 4. In some embodiments, at least 90% of the ALK1-Fc fusion protein is in a dimeric form. In some embodiments, the ALK1-ECD polypeptide and/or the ALK1-Fc fusion protein is glycosylated. In some embodiments, the ALK1-Fc fusion protein is Dalantercept. In some embodiments, the ALK1-ECD polypeptide is administered to the subject at a dosage of 0.1-30 mg/kg/day. In some embodiments, the head and neck cancer is a recurrent or metastatic squamous cell carcinoma.

Some aspects of this disclosure provide pharmaceutical compositions for the treatment of head and neck cancer in a subject. In some embodiments, the composition comprises (a) an agent selected from the group consisting of (i) an ALK1-extracellular domain (ALK1-ECD) polypeptide; (ii) an antibody that binds to BMP9 or BMP10; or (iii) an antibody that binds to an ALK1 polypeptide comprising amino acids 22-118 of SEQ ID NO: 1; and (b) a chemotherapeutic platinum agent, wherein the agent of (a) and the chemotherapeutic platinum agent are in an amount sufficient to treat a head and neck cancer in the subject. In some embodiments, the amino acid sequence of the ALK1-ECD polypeptide is at least 90% identical to the sequence of amino acids 22-118 of SEQ ID NO: 1. In some embodiments, the amino acid sequence of the ALK1-ECD polypeptide is at least 97% identical to the sequence of amino acids 22-118 of SEQ ID NO: 1. In some embodiments, the C-terminal amino acid residue of the ALK1-ECD polypeptide is not glutamine 118 (Q118) of SEQ ID NO: 1. In some embodiments, the C-terminal amino acid residue of the ALK1-ECD polypeptide is not a glutamine residue. In some embodiments, the C-terminal amino acid residue of the ALK1-ECD polypeptide is proline 113 (P113), glycine 114 (G114), threonine 115 (T115), aspartic acid 116 (D116), glycine 117 (G117), leucine 119 (L119), alanine 120 (A120), leucine 121 (L121), isoleucine 122 (I122), or leucine 123 (L123) of SEQ ID NO: 1. In some embodiments, the ALK1-ECD polypeptide is fused to an Fc portion of an immunoglobulin thus forming an ALK1-Fc fusion protein. In some embodiments, the Fc portion is an Fc portion of a human IgG1. In some embodiments, the ALK1-Fc fusion protein comprises the sequence of SEQ ID NO: 3 or SEQ ID NO: 4. In some embodiments, the amino acid sequence of the ALK1-Fc fusion protein is at least 90% identical to the amino acid sequence of SEQ ID NO: 3 or SEQ ID NO: 4. In some embodiments, the amino acid sequence of the ALK1-Fc fusion protein is at least 97% identical to the amino acid sequence of SEQ ID NO: 3 or SEQ ID NO: 4. In some embodiments, the ALK1-ECD polypeptide and/or the ALK1-Fc fusion protein binds BMP9 or BMP10 with a K_{D} of less than 1×10⁻⁷M, e.g., with a K_{D} of less than 1×10⁻⁸M, less than 1×10⁻⁹M, less than 1×10⁻¹⁰M, or less than 1×10⁻¹¹M. In some embodiments, the ALK1-ECD polypeptide and/or the ALK1-Fc fusion protein binds to TGFβ-1 with a K_{D} of greater than 1×10⁻⁶M. In some embodiments, the ALK1-ECD polypeptide is administered in a pharmaceutical preparation wherein at least 90% of the ALK1-Fc fusion protein is in a dimeric form.

In some embodiments, the antibody of (ii) binds to an ALK1-ECD polypeptide with a K_{D} of less than 5×10⁻⁸ M. In some embodiments, the antibody of (ii) binds to an ALK1-ECD polypeptide with a K_{D} of less than 1×10⁻¹⁰ M. In some embodiments, the antibody of (ii) inhibits angiogenesis stimulated by an ALK1 ligand. In some embodiments, the antibody of (ii) inhibits the binding of BMP9 or BMP10 to an ALK1-ECD polypeptide. In some embodiments, the antibody of (iii) binds to an ALK1-ECD polypeptide with a K_{D} of less than 5×10⁻⁸ M. In some embodiments, the antibody of (iii) inhibits angiogenesis stimulated by at least one ALK1 ligand.

In some embodiments, the chemotherapeutic platinum agent comprises a coordination complex of platinum. In some embodiments, the chemotherapeutic platinum agent is cisplatin, carboplatin, oxaliplatin, satraplatin, picoplatin, Nedaplatin, Triplatin, Lipoplatin, or any combination thereof. In some embodiments, the head and neck cancer is a recurrent or metastatic squamous cell carcinoma.

The summary above is meant to illustrate, in a non-limiting manner, some of the embodiments, advantages, features, and uses of the technology disclosed herein. Other embodiments, advantages, features, and uses of the technology disclosed herein will be apparent from the Detailed Description, the Drawings, the Examples, and the Claims.

### BRIEF DESCRIPTION OF THE DRAWINGS

FIG. 1. Tumor growth. Comparison of control, ACE-041-treatment, cisplatin-treatment and combination treatment.

FIG. 2. Changes in animal body weight. Comparison of control, ACE-041-treatment, cisplatin-treatment and combination treatment.

FIG. 3. Changes in individual body weight at the end of the study. Comparison of control, ACE-041-treatment, cisplatin-treatment and combination treatment.

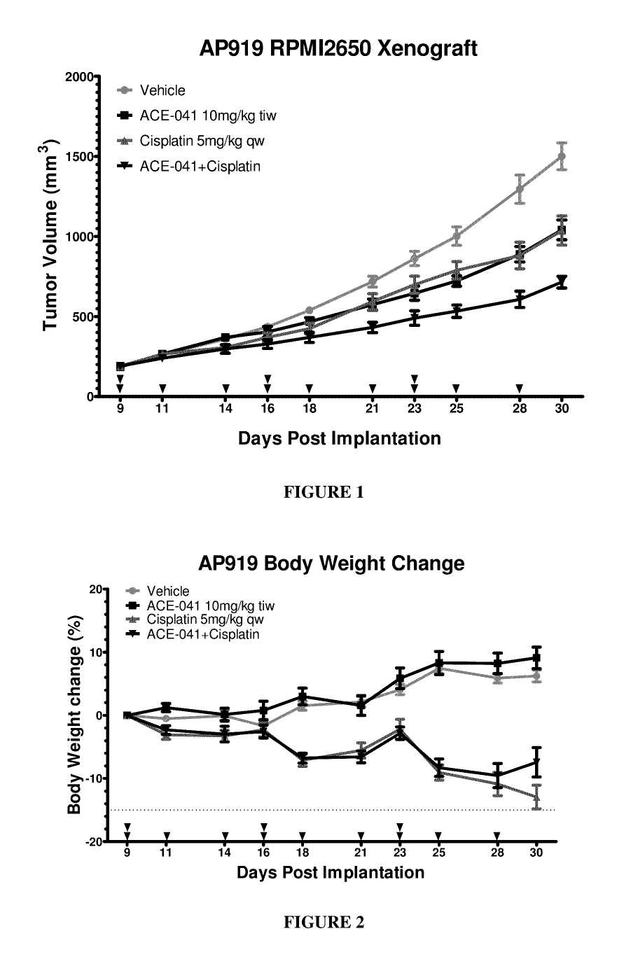

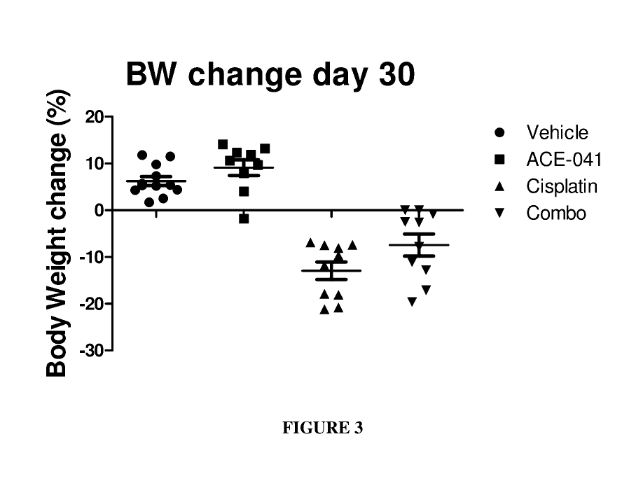

### DETAILED DESCRIPTION

Some aspects of this disclosure relate to the surprising discovery that the effectiveness of ALK1 inhibitors in the treatment of cancer can be further improved by using such inhibitors in combination with chemotherapeutic platinum agents, while some other chemotherapeutic agents did not show such a synergistic effect when used in combination with ALK1 inhibitors. Some aspects of this disclosure relate to the surprising discovery that some types of cancer, e.g., human papilloma virus (HPV)-positive cancers, are particularly sensitive to treatment with ALK1 inhibitors. The compositions and methods provided herein are thus particularly useful in the treatment of cancer with combination therapies of ALK1 inhibitors and chemotherapeutic platinum agents as well as in the diagnosis, selection for treatment, prognosis, and treatment of cancers with ALK1 inhibitors.

### Introduction

ALK1 is a type I cell-surface receptor for the TGF-(3 superfamily of ligands and is also known as ACVRL1 and ACVRLK1. ALK1 has been implicated as a receptor for TGF-β1, TGF-β3 and BMP-9 (Marchuk et al., Hum Mol Genet. 2003; Brown et al., J Biol Chem. 2005 Jul. 1;280(26):25111-8). In mice, loss-of-function mutations in ALK1 lead to a variety of abnormalities in the developing vasculature (Oh et al., Proc. Natl Acad. Sci. USA 2000, 97, 2626-2631; Urness et al., Nat. Genet. 2000, 26, 328-331).

In humans, loss-of-function mutations in ALK1 are associated with hereditary hemorrhagic telangiectasia (HHT, or Osler-Rendu-Weber syndrome), in which patients develop arteriovenous malformations that create direct flow (communication) from an artery to a vein (arteriovenous shunt), without an intervening capillary bed. Typical symptoms of patients with HHT include recurrent epistaxis, gastrointestinal hemorrhage, cutaneous and mucocutaneous telangiectases, and arteriovenous malformations (AVM) in the pulmonary, cerebral, or hepatic vasculature.

Some aspects of this disclosure relate to the discovery that ALK1 inhibitors can be used to effectively treat cancer. Some aspects of this disclosure relate to the discovery that ALK1 inhibitors are particularly effective in the treatment of head and neck cancer. Some aspects of this disclosure relate to the discovery that ALK1 inhibitors can be used to treat a variety of cancers when used in combination with other chemotherapeutic agents, such as chemotherapeutic platinum agents.

The mechanism of action of ALK1 has been delineated previously. For example, the identity of physiological, high affinity ligands for ALK1 (e.g., BMP9 and BMP10) has been described and it has been demonstrated that ALK1 ECD polypeptides inhibit angiogenesis in vitro and in vivo (see, e.g., U.S. Pat. No.8,455,428, the entire contents of which are incorporated herein by reference). ALK1 ECD polypeptides can exert an anti-angiogenic effect even if the ALK1 ECD polypeptides do not exhibit meaningful or substantial binding to TGF-β1 (e.g., if they bind TGF-β1 with a K_{D}>10⁻⁶). Moreover, ALK1 ECD polypeptides inhibit angiogenesis that is stimulated by many different pro-angiogenic factors, including VEGF, FGF, and GDF7. Thus, polypeptides comprising a portion of the extracellular domain of ALK1 (“ALK1 ECD polypeptides”) may be used to inhibit angiogenesis in vivo, including VEGF-independent angiogenesis and angiogenesis that is mediated by multiple angiogenic factors, including VEGF, FGF and PDGF, and thus may be used to treat cancers associated with or dependent upon such types of angiogenesis.

Naturally occurring ALK1 proteins are transmembrane proteins, with a portion of the protein positioned outside the cell (the extracellular portion) and a portion of the protein positioned inside the cell (the intracellular portion). Aspects of the present disclosure encompass polypeptides comprising a portion of the extracellular domain of ALK1.

### ALK1-ECD Polypeptides and ALK1-Fc Fusion Proteins

The term “ALK1 ECD polypeptide,” as used herein, refers to a polypeptide consisting of or comprising an amino acid sequence of an extracellular domain of a naturally occurring ALK1 polypeptide, either including or excluding any signal sequence and sequence N-terminal to the signal sequence, or an amino acid sequence that is at least 33 percent identical to an extracellular domain of a naturally occurring ALK1 polypeptide, and optionally at least 60%, at least 70%, at least 80%, at least 85%, at least 90%, at least 95%, at least 96%, at least 97%, at least 98%, at least 99% or 100% identical to the sequence of an extracellular domain of a naturally occurring ALK1 polypeptide, as exemplified by the cysteine knot region of amino acids 34-95 of SEQ ID NO:1 or the cysteine knot plus additional amino acids at the N- and C-termini of the extracellular domain, such as amino acids 22-118 of SEQ ID NO.: 1. Likewise, an ALK1 ECD polypeptide may comprise a polypeptide that is encoded by nucleotides 100-285 of SEQ ID NO: 2, or silent variants thereof or nucleic acids that hybridize to the complement thereof under stringent hybridization conditions (generally, such conditions are known in the art but may, for example, involve hybridization in 50% v/v formamide, 5×SSC, 2% w/v blocking agent, 0.1% N-lauroylsarcosine, 0.3% SDS at 65C.° overnight and washing in, for example, 5×SSC at about 65° C. Additionally, an ALK1 ECD polypeptide may comprise a polypeptide that is encoded by nucleotides 64-384 of SEQ ID NO: 2, or silent variants thereof or nucleic acids that hybridize to the complement thereof under stringent hybridization conditions (generally, such conditions are known in the art but may, for example, involve hybridization in 50% v/v formamide, 5×SSC, 2% w/v blocking agent, 0.1% N-lauroylsarcosine, 0.3% SDS at 65° C. overnight and washing in, for example, 5×SSC at about 65° C.

The term “ALK1 ECD polypeptide” accordingly encompasses isolated extracellular portions of ALK1 polypeptides, variants thereof (including variants that comprise, for example, no more than 1, 2, 3, 4, 5, 6, 7, 8, 9, or 10 amino acid substitutions, additions or deletions in the sequence corresponding to amino acids 22-118 of SEQ ID NO: 1 and including variants that comprise no more than 1, 2, 3, 4, 5, 6, 7, 8, 9, or 10 amino acid substitutions, additions or deletions in the sequence corresponding to amino acids 34-95 of SEQ ID NO: 1), fragments thereof and fusion proteins comprising any of the preceding. Preferably, any of the foregoing ALK1 ECD polypeptides will retain substantial affinity for BMP9 or BMP10. The term “ALK1 ECD polypeptide” is explicitly intended to exclude any full-length, naturally occurring ALK1 polypeptide. Generally, an ALK1 ECD polypeptide will be designed to be soluble in aqueous solutions at biologically relevant temperatures, pH levels and osmolarity.

As described above, the disclosure provides ALK1 ECD polypeptides sharing a specified degree of sequence identity or similarity to a naturally occurring ALK1 polypeptide. Methods for determining sequence identity are well known to those of skill in the art, and such methods typically include aligning two or more sequences and quantifying the percentage of amino acid residues that are identical in the aligned sequences. For optimal comparison purposes, gaps can be introduced in one or both of a first and a second amino acid or nucleic acid sequence and non-homologous sequences can be disregarded. Exemplary suitable methods and strategies of determining and quantifying sequence identity are described in U.S. Pat. No. 8,455,428, the entire contents of which are incorporated herein by reference. Additional suitable methods will be apparent to the skilled artisan.

In some embodiments, ALK1 ECD polypeptides comprise an extracellular portion of a naturally occurring ALK1 protein such as a sequence of SEQ ID NO: 1, and preferably a ligand binding portion of the ALK1 extracellular domain. In certain embodiments, a soluble ALK1 polypeptide comprises an amino acid sequence that is at least 60%, 70%, 80%, 85%, 90%, 95%, 96%, 97%, 98% or 99% identical to an amino acid sequence of amino acids 22-118 of the SEQ ID NO: 1. In certain embodiments, a truncated extracellular ALK1 polypeptide comprises at least 30, 40 or 50 consecutive amino acids of an amino acid sequence of an extracellular portion of SEQ ID NO: 1.

In some embodiments, an ALK1 ECD polypeptide binds to one or both of BMP9 and BMP10. Optionally the ALK1 polypeptide does not show substantial binding to TGF-β1 or TGF-β3, e.g., in that the ALK1 polypeptide binds TGF-β1 or TGF-β3 with a K_{D}>10⁻⁶. Binding may be assessed using purified proteins in solution or in a surface plasmon resonance system, such as a Biacore™ system.

Preferred soluble ALK1 polypeptides will exhibit an anti-angiogenic activity. Bioassays for angiogenesis inhibitory activity include the chick chorioallantoic membrane (CAM) assay, the mouse corneal micropocket assay, an assay for measuring the effect of administering isolated or synthesized proteins on implanted tumors. The CAM assay is described by O'Reilly, et al. in “Angiogenic Regulation of Metastatic Growth” Cell, vol. 79 (2), Oct. 1, 1994, pp. 315-328. Briefly, 3 day old chicken embryos with intact yolks are separated from the egg and placed in a petri dish. After 3 days of incubation, a methylcellulose disc containing the protein to be tested is applied to the CAM of individual embryos. After 48 hours of incubation, the embryos and CAMs are observed to determine whether endothelial growth has been inhibited. The mouse corneal micropocket assay involves implanting a growth factor-containing pellet, along with another pellet containing the suspected endothelial growth inhibitor, in the cornea of a mouse and observing the pattern of capillaries that are elaborated in the cornea. Other suitable assays are described in U.S. Pat. No. 8,455,428, the entire contents of which are incorporated herein by reference, and additional suitable assays will be apparent to those of skill in the art.

ALK1 ECD polypeptides may be produced by removing the cytoplasmic tail and the transmembrane region of an ALK1 polypeptide. Alternatively, the transmembrane domain may be inactivated by deletion, or by substitution of the normally hydrophobic amino acid residues which comprise a transmembrane domain with hydrophilic ones. In either case, a substantially hydrophilic hydropathy profile is created which will reduce lipid affinity and improve aqueous solubility. Deletion of the transmembrane domain is preferred over substitution with hydrophilic amino acid residues because it avoids introducing potentially immunogenic epitopes.

ALK1 ECD polypeptides may additionally include any of various leader sequences at the N-terminus. Such a sequence would allow the peptides to be expressed and targeted to the secretion pathway in a eukaryotic system. See, e.g., Ernst et al., U.S. Pat. No. 5,082,783 (1992). Alternatively, a native ALK1 signal sequence may be used to effect extrusion from the cell. Possible leader sequences include native, tPa and honeybee mellitin leaders (SEQ ID NOs.: 7-9, respectively). Processing of signal peptides may vary depending on the leader sequence chosen, the cell type used and culture conditions, among other variables, and therefore actual N-terminal start sites for mature ALK1 ECD polypeptides, including that of SEQ ID NO: 1, may shift by 1-5 amino acids in either the N-terminal or C-terminal direction.

In certain embodiments, the present disclosure contemplates specific mutations of the ALK1 polypeptides so as to alter the glycosylation of the polypeptide. Such mutations may be selected so as to introduce or eliminate one or more glycosylation sites, such as O-linked or N-linked glycosylation sites. Asparagine-linked glycosylation recognition sites generally comprise a tripeptide sequence, asparagine-X-threonine (or asparagines-X-serine) (where “X” is any amino acid) which is specifically recognized by appropriate cellular glycosylation enzymes. The alteration may also be made by the addition of, or substitution by, one or more serine or threonine residues to the sequence of the wild-type ALK1 polypeptide (for O-linked glycosylation sites). A variety of amino acid substitutions or deletions at one or both of the first or third amino acid positions of a glycosylation recognition site (and/or amino acid deletion at the second position) results in non-glycosylation at the modified tripeptide sequence. Another means of increasing the number of carbohydrate moieties on an ALK1 polypeptide is by chemical or enzymatic coupling of glycosides to the ALK1 polypeptide. Depending on the coupling mode used, the sugar(s) may be attached to (a) arginine and histidine; (b) free carboxyl groups; (c) free sulfhydryl groups such as those of cysteine; (d) free hydroxyl groups such as those of serine, threonine, or hydroxyproline; (e) aromatic residues such as those of phenylalanine, tyrosine, or tryptophan; or (f) the amide group of glutamine. These methods are described in WO 87/05330 published Sep. 11, 1987, and in Aplin and Wriston (1981) CRC Crit. Rev. Biochem., pp. 259-306, incorporated by reference herein. Removal of one or more carbohydrate moieties present on an ALK1 polypeptide may be accomplished chemically and/or enzymatically. Chemical deglycosylation may involve, for example, exposure of the ALK1 polypeptide to the compound trifluoromethanesulfonic acid, or an equivalent compound. This treatment results in the cleavage of most or all sugars except the linking sugar (N-acetylglucosamine or N-acetylgalactosamine), while leaving the amino acid sequence intact. Chemical deglycosylation is further described by Hakimuddin et al. (1987) Arch. Biochem. Biophys. 259:52 and by Edge et al. (1981) Anal. Biochem. 118:131. Enzymatic cleavage of carbohydrate moieties on ALK1 polypeptides can be achieved by the use of a variety of endo- and exo-glycosidases as described by Thotakura et al. (1987) Meth. Enzymol. 138:350. The sequence of an ALK1 polypeptide may be adjusted, as appropriate, depending on the type of expression system used, as mammalian, yeast, insect and plant cells may all introduce differing glycosylation patterns that can be affected by the amino acid sequence of the peptide. In general, ALK1 proteins for use in humans will be expressed in a mammalian cell line that provides proper glycosylation, such as HEK293 or CHO cell lines, although other mammalian expression cell lines, yeast cell lines with engineered glycosylation enzymes and insect cells are expected to be useful as well.

This disclosure further contemplates a method of generating mutants, particularly sets of combinatorial mutants of an ALK1 polypeptide, as well as truncation mutants; pools of combinatorial mutants are especially useful for identifying functional variant sequences. The purpose of screening such combinatorial libraries may be to generate, for example, ALK1 polypeptide variants which can act as either agonists or antagonist, or alternatively, which possess novel activities all together. A variety of screening assays are provided below, and such assays may be used to evaluate variants. For example, an ALK1 polypeptide variant may be screened for ability to bind to an ALK1 ligand, to prevent binding of an ALK1 ligand to an ALK1 polypeptide or to interfere with signaling caused by an ALK1 ligand. The activity of an ALK1 polypeptide or its variants may also be tested in a cell-based or in vivo assay, particularly any of the assays disclosed in the Examples.

In certain embodiments, the ALK1 ECD polypeptides of the disclosure may further comprise post-translational modifications in addition to any that are naturally present in the ALK1 polypeptides. Such modifications include, but are not limited to, acetylation, carboxylation, glycosylation, phosphorylation, lipidation, and acylation. As a result, the modified ALK1 ECD polypeptides may contain non-amino acid elements, such as polyethylene glycols, lipids, poly- or mono-saccharide, and phosphates. Effects of such non-amino acid elements on the functionality of an ALK1 ECD polypeptide may be tested as described herein for other ALK1 ECD polypeptide variants. When an ALK1 ECD polypeptide is produced in cells by cleaving a nascent form of the ALK1 polypeptide, post-translational processing may also be important for correct folding and/or function of the protein. Different cells (such as CHO, HeLa, MDCK, 293, WI38, NIH-3T3 or HEK293) have specific cellular machinery and characteristic mechanisms for such post-translational activities and may be chosen to ensure the correct modification and processing of the ALK1 polypeptides.

In certain aspects, functional variants or modified forms of the ALK1 ECD polypeptides include fusion proteins having at least a portion of the ALK1 ECD polypeptides and one or more fusion domains. Well known examples of such fusion domains include, but are not limited to, polyhistidine, Glu-Glu, glutathione S transferase (GST), thioredoxin, protein A, protein G, an immunoglobulin heavy chain constant region (Fc), maltose binding protein (MBP), or human serum albumin. A fusion domain may be selected so as to confer a desired property. For example, some fusion domains are particularly useful for isolation of the fusion proteins by affinity chromatography. For the purpose of affinity purification, relevant matrices for affinity chromatography, such as glutathione-, amylase-, and nickel- or cobalt-conjugated resins are used. Many of such matrices are available in “kit” form, such as the Pharmacia GST purification system and the QIAexpress™ system (Qiagen) useful with (HIS₆) fusion partners. As another example, a fusion domain may be selected so as to facilitate detection of the ALK1 ECD polypeptides. Examples of such detection domains include the various fluorescent proteins (e.g., GFP) as well as “epitope tags,” which are usually short peptide sequences for which a specific antibody is available. Well known epitope tags for which specific monoclonal antibodies are readily available include FLAG, influenza virus haemagglutinin (HA), and c-myc tags. In some cases, the fusion domains have a protease cleavage site, such as for Factor Xa or Thrombin, which allows the relevant protease to partially digest the fusion proteins and thereby liberate the recombinant proteins therefrom. The liberated proteins can then be isolated from the fusion domain by subsequent chromatographic separation. In certain preferred embodiments, an ALK1 ECD polypeptide is fused with a domain that stabilizes the ALK1 polypeptide in vivo (a “stabilizer” domain). By “stabilizing” is meant anything that increases serum half-life, regardless of whether this is because of decreased destruction, decreased clearance by the kidney, or other pharmacokinetic effect. Fusions with the Fc portion of an immunoglobulin are known to confer desirable pharmacokinetic properties on a wide range of proteins Likewise, fusions to human serum albumin can confer desirable properties. Other types of fusion domains that may be selected include multimerizing (e.g., dimerizing, tetramerizing) domains and functional domains.

For example, the present disclosure provides a fusion protein comprising a soluble extracellular domain of ALK1 fused to an Fc domain. An exemplary amino acid sequence of such a domain is provided in SEQ ID NO: 5.

In some embodiments, the Fc domain has one or more mutations at residues such as Asp-265, lysine 322, and Asn-434. In certain cases, the mutant Fc domain having one or more of these mutations (e.g., Asp-265 mutation) has reduced ability of binding to the Fc receptor relative to a wild type Fc domain. In other cases, the mutant Fc domain having one or more of these mutations (e.g., Asn-434 mutation) has increased ability of binding to the MHC class I-related Fc-receptor (FcRN) relative to a wild type Fc domain.

It is understood that different elements of the fusion proteins may be arranged in any manner that is consistent with the desired functionality. For example, an ALK1 ECD polypeptide may be placed C-terminal to a heterologous domain, or, alternatively, a heterologous domain may be placed C-terminal to an ALK1 ECD polypeptide. The ALK1 ECD polypeptide domain and the heterologous domain need not be adjacent in a fusion protein, and additional domains or amino acid sequences may be included C- or N-terminal to either domain or between the domains.

As used herein, the term, “immunoglobulin Fc region” or simply “Fc” is understood to mean the carboxyl-terminal portion of an immunoglobulin chain constant region, preferably an immunoglobulin heavy chain constant region, or a portion thereof. For example, an immunoglobulin Fc region may comprise 1) a CH1 domain, a CH2 domain, and a CH3 domain, 2) a CH1 domain and a CH2 domain, 3) a CH1 domain and a CH3 domain, 4) a CH2 domain and a CH3 domain, or 5) a combination of two or more domains and an immunoglobulin hinge region. In a preferred embodiment the immunoglobulin Fc region comprises at least an immunoglobulin hinge region a CH2 domain and a CH3 domain, and preferably lacks the CH1 domain.

In some embodiments, the class of immunoglobulin from which the heavy chain constant region is derived is IgG (Igγ) (γ subclasses 1, 2, 3, or 4). Other classes of immunoglobulin, IgA (Igα), IgD (Igδ), IgE (Igε) and IgM (Igμ), may be used. The choice of appropriate immunoglobulin heavy chain constant region is discussed in detail in U.S. Pat. Nos. 5,541,087, and 5,726,044. The choice of particular immunoglobulin heavy chain constant region sequences from certain immunoglobulin classes and subclasses to achieve a particular result is considered to be within the level of skill in the art. The portion of the DNA construct encoding the immunoglobulin Fc region preferably comprises at least a portion of a hinge domain, and preferably at least a portion of a CH₃ domain of Fc γ or the homologous domains in any of IgA, IgD, IgE, or IgM.

Furthermore, it is contemplated that substitution or deletion of amino acids within the immunoglobulin heavy chain constant regions may be useful in the practice of the methods and compositions disclosed herein. One example would be to introduce amino acid substitutions in the upper CH2 region to create an Fc variant with reduced affinity for Fc receptors (Cole et al. (1997) J. Immunol. 159:3613).

In certain embodiments, the present disclosure makes available isolated and/or purified forms of the ALK1 ECD polypeptides, which are isolated from, or otherwise substantially free of (e.g., at least 80%, 90%, 95%, 96%, 97%, 98% or 99% free of), other proteins and/or other ALK1 ECD polypeptide species. In some embodiments, ALK1 polypeptides will generally be produced by expression from recombinant nucleic acids in a suitable host cell. The host cell may be any prokaryotic or eukaryotic cell. For example, a polypeptide of the present disclosure may be expressed in bacterial cells such as E. coli, insect cells (e.g., using a baculovirus expression system), yeast, or mammalian cells. Other suitable host cells are known to those skilled in the art. Accordingly, some embodiments of the present disclosure further pertain to methods of producing the ALK1 ECD polypeptides. It has been established that an ALK1-Fc fusion protein set forth in SEQ ID NO:3 or SEQ ID NO: 4 and expressed in CHO cells has potent anti-angiogenic activity.

In some embodiments, the ALK1-ECD polypeptide is a soluble fusion protein containing the extracellular domain of activin receptor-like kinase-1 (ALK1) fused to a human Fc domain (ALK1-Fc fusion protein). In some embodiments, the amino acid sequence of the ALK1-Fc fusion protein comprises or consists of the sequence as provided in SEQ ID NO: 3 or SEQ ID NO: 4. In particular embodiments, an ALK1-inhibitory antibody (e.g., an antibody that inhibits ALK1 signaling, e.g., by binding ALK1 or an ALK1 ligand and inhibiting an interaction between ALK1 and an ALK1 ligand) or ALK1-Fc fusion protein may be modified to either enhance or inhibit complement dependent cytotoxicity (CDC). Modulated CDC activity may be achieved by introducing one or more amino acid substitutions, insertions, or deletions in an Fc region (see, e.g., U.S. Pat. No. 6,194,551). Alternatively or additionally, cysteine residue(s) may be introduced in the Fc region, thereby allowing interchain disulfide bond formation in this region. The homodimeric ALK1-Fc fusion protein or ALK1-inhibitory antibody thus generated may have improved or reduced internalization capability and/or increased or decreased complement-mediated cell killing. See Caron et al., J. Exp Med. 176:1191-1195 (1992) and Shopes, B. J. Immunol. 148:2918-2922 (1992), W099/51642, Duncan & Winter Nature 322: 738-40 (1988); U.S. Pat. No. 5,648,260; U.S. Pat. No. 5,624,821; and WO94/29351.

In some embodiments, one or more of the following intramolecular or intermolecular disulfide bridges exist between two ALK1-Fc fusion protein monomers in the dimeric form: 13-30, 13′-30′, 15-20, 15′-20′, 25-48, 25′-48′, 56-68, 56′-68′, 69-74, 69′-74′, 107-107′, 110-110′, 142-202, 142′-202′, 248-306, and 248′-306′, wherein amino acids in the first monomer are designated without prime (′) while amino acid residues in the second monomer are designated with a prime. In some embodiments, the ALK1-Fc fusion protein is Dalantercept, which is also sometimes referred to as ACE-041. See SEQ ID NO: 3 for an exemplary amino acid sequence.

### Antibodies

Another aspect of the disclosure pertains to an antibody reactive with an extracellular portion of an ALK1 polypeptide, preferably antibodies that are specifically reactive with ALK1 polypeptide. In a preferred embodiment, such antibody interferes with ALK1 binding to a BMP-9 and/or BMP-10 ligand. It will be understood that an antibody against a ligand of ALK1 should bind to the mature, processed form of the relevant protein. The disclosure also provides antibodies that bind to ALK1 ligands, including, but not limited to, antibodies that bind to GDF5, GDF6, GDF7, BMP9 and/or BMP10, and that inhibit ALK1 binding to such ligands. Preferred antibodies will exhibit an anti-angiogenic activity in a bioassay, such as a CAM assay or corneal micropocket assay.

The term “antibody” as used herein is intended to include whole antibodies, e.g., of any isotype (IgG, IgA, IgM, IgE, etc), and includes fragments or domains of immunoglobulins which are reactive with a selected antigen. Antibodies can be fragmented using conventional techniques and the fragments screened for utility and/or interaction with a specific epitope of interest. Thus, the term includes segments of proteolytically-cleaved or recombinantly-prepared portions of an antibody molecule that are capable of selectively reacting with a certain protein. Non-limiting examples of such proteolytic and/or recombinant fragments include Fab, F(ab′)2, Fab′ , Fv, and single chain antibodies (scFv) containing a V[L] and/or V[H] domain joined by a peptide linker. The scFv's may be covalently or non-covalently linked to form antibodies having two or more binding sites. The term antibody also includes polyclonal, monoclonal, or other purified preparations of antibodies and recombinant antibodies. The term “recombinant antibody”, means an antibody, or antigen binding domain of an immunoglobulin, expressed from a nucleic acid that has been constructed using the techniques of molecular biology, such as a humanized antibody or a fully human antibody developed from a single chain antibody. Single domain and single chain antibodies are also included within the term “recombinant antibody”.

Antibodies may be generated by any of the various methods known in the art, including administration of antigen to an animal, administration of antigen to an animal that carries human immunoglobulin genes, or screening with an antigen against a library of antibodies (often single chain antibodies or antibody domains). Once antigen binding activity is detected, the relevant portions of the protein may be grafted into other antibody frameworks, including full-length IgG frameworks. For example, by using immunogens derived from an ALK1 polypeptide or an ALK1 ligand, anti-protein/anti-peptide antisera or monoclonal antibodies can be made by standard protocols (See, for example, Antibodies: A Laboratory Manual ed. by Harlow and Lane (Cold Spring Harbor Press: 1988)). A mammal, such as a mouse, a hamster or rabbit can be immunized with an immunogenic form of the peptide (e.g., a ALK1 polypeptide or an antigenic fragment which is capable of eliciting an antibody response, or a fusion protein). Techniques for conferring immunogenicity on a protein or peptide include conjugation to carriers or other techniques well known in the art. An immunogenic portion (preferably an extracellular portion) of an ALK1 polypeptide can be administered in the presence of adjuvant. The progress of immunization can be monitored by detection of antibody titers in plasma or serum. Standard ELISA or other immunoassays can be used with the immunogen as antigen to assess the levels of antibodies.

Following immunization of an animal with an antigenic preparation of an ALK1 polypeptide, anti-ALK1 antisera can be obtained and, if desired, polyclonal anti-ALK1 antibodies can be isolated from the serum. To produce monoclonal antibodies, antibody-producing cells (lymphocytes) can be harvested from an immunized animal and fused by standard somatic cell fusion procedures with immortalizing cells such as myeloma cells to yield hybridoma cells. Such techniques are well known in the art, and include, for example, the hybridoma technique (originally developed by Kohler and Milstein, (1975) Nature, 256: 495-497), the human B cell hybridoma technique (Kozbar et al., (1983) Immunology Today, 4: 72), and the EBV-hybridoma technique to produce human monoclonal antibodies (Cole et al., (1985) Monoclonal Antibodies and Cancer Therapy, Alan R. Liss, Inc. pp. 77-96). Hybridoma cells can be screened immunochemically for production of antibodies specifically reactive with a mammalian ALK1 polypeptide of the present disclosure and monoclonal antibodies isolated from a culture comprising such hybridoma cells.

The term antibody as used herein is intended to include fragments thereof which are also specifically reactive with one of the subject ALK1 polypeptides. Antibodies can be fragmented using conventional techniques and the fragments screened for utility in the same manner as described above for whole antibodies. For example, F(ab)₂ fragments can be generated by treating antibody with pepsin. The resulting F(ab)₂ fragment can be treated to reduce disulfide bridges to produce Fab fragments. The antibody of the present disclosure is further intended to include bispecific, single-chain, and chimeric and humanized molecules having affinity for an ALK1 polypeptide conferred by at least one CDR region of the antibody. In preferred embodiments, the antibody further comprises a label attached thereto and is able to be detected, (e.g., the label can be a radioisotope, fluorescent compound, enzyme or enzyme co-factor).

In certain preferred embodiments, an antibody of the disclosure is a recombinant antibody, particularly a humanized monoclonal antibody or a fully human recombinant antibody.

The term “specifically reactive with” as used in reference to an antibody is intended to mean, as is generally understood in the art, that the antibody is sufficiently selective between the antigen of interest (e.g. an ALK1 polypeptide or an ALK1 ligand) and other antigens that are not of interest that the antibody is useful for, at minimum, detecting the presence of the antigen of interest in a particular type of biological sample. In certain methods employing the antibody, a higher degree of specificity in binding may be desirable. For example, an antibody for use in detecting a low abundance protein of interest in the presence of one or more very high abundance protein that are not of interest may perform better if it has a higher degree of selectivity between the antigen of interest and other cross-reactants. Monoclonal antibodies generally have a greater tendency (as compared to polyclonal antibodies) to discriminate effectively between the desired antigens and cross-reacting polypeptides. In addition, an antibody that is effective at selectively identifying an antigen of interest in one type of biological sample (e.g. a stool sample) may not be as effective for selectively identifying the same antigen in a different type of biological sample (e.g. a blood sample) Likewise, an antibody that is effective at identifying an antigen of interest in a purified protein preparation that is devoid of other biological contaminants may not be as effective at identifying an antigen of interest in a crude biological sample, such as a blood or urine sample. Accordingly, in preferred embodiments, the application provides antibodies that have demonstrated specificity for an antigen of interest in a sample type that is likely to be the sample type of choice for use of the antibody.

One characteristic that influences the specificity of an antibody:antigen interaction is the affinity of the antibody for the antigen. Although the desired specificity may be reached with a range of different affinities, generally preferred antibodies will have an affinity (a dissociation constant) of about 10⁻⁶, 10⁻⁷, 10⁻⁸, 10⁻⁹ or less. Given the apparently low binding affinity of TGFβ for ALK1, it is expected that many anti-ALK1 antibodies will inhibit TGFβ binding. However, the GDF5, 6, 7 group of ligands bind with a K_{D} of approximately 5×10⁻⁸ M and the BMP 9,10 ligands bind with a K_{D} of approximately 1×10⁻¹⁰ M. Thus, antibodies of appropriate affinity may be selected to interfere with the signaling activities of these ligands.

A variety of different techniques are available for testing antibody:antigen interactions to identify particularly desirable antibodies. Such techniques include ELISAs, surface plasmon resonance binding assays (e.g. the Biacore binding assay, Bia-core AB, Uppsala, Sweden), sandwich assays (e.g. the paramagnetic bead system of IGEN International, Inc., Gaithersburg, Md.), western blots, immunoprecipitation assays and immunohistochemistry.

The application further provides antibodies and ALK1-Fc fusion proteins a with engineered or variant Fc regions. Such antibodies and Fc fusion proteins may be useful, for example, in modulating effector functions, such as, antigen-dependent cytotoxicity (ADCC) and complement-dependent cytotoxicity (CDC). Additionally, the modifications may improve the stability of the antibodies and Fc fusion proteins. Amino acid sequence variants of the antibodies and Fc fusion proteins are prepared by introducing appropriate nucleotide changes into the DNA, or by peptide synthesis. Such variants include, for example, deletions from, and/or insertions into and/or substitutions of, residues within the amino acid sequences of the antibodies and Fc fusion proteins disclosed herein. Any combination of deletion, insertion, and substitution is made to arrive at the final construct, provided that the final construct possesses the desired characteristics. The amino acid changes also may alter post-translational processes of the antibodies and Fc fusion proteins, such as changing the number or position of glycosylation sites.

Antibodies and Fc fusion proteins with reduced effector function may be produced by introducing changes in the amino acid sequence, including, but are not limited to, the Ala-Ala mutation described by Bluestone et al. (see WO 94/28027 and WO 98/47531; also see Xu et al. 2000 Cell Immunol 200; 16-26). Thus in certain embodiments, antibodies and Fc fusion proteins of the disclosure with mutations within the constant region including the Ala-Ala mutation may be used to reduce or abolish effector function. According to these embodiments, antibodies and Fc fusion proteins may comprise a mutation to an alanine at position 234 or a mutation to an alanine at position 235, or a combination thereof. In one embodiment, the antibody or Fc fusion protein comprises an IgG4 framework, wherein the Ala-Ala mutation would describe a mutation(s) from phenylalanine to alanine at position 234 and/or a mutation from leucine to alanine at position 235. In another embodiment, the antibody or Fc fusion protein comprises an IgG 1 framework, wherein the Ala-Ala mutation would describe a mutation(s) from leucine to alanine at position 234 and/or a mutation from leucine to alanine at position 235. The antibody or Fc fusion protein may alternatively or additionally carry other mutations, including the point mutation K322A in the CH2 domain (Hezareh et al. 2001 J Virol. 75: 12161-8).

In particular embodiments, the antibody or Fc fusion protein may be modified to either enhance or inhibit complement dependent cytotoxicity (CDC). Modulated CDC activity may be achieved by introducing one or more amino acid substitutions, insertions, or deletions in an Fc region (see, e.g., U.S. Pat. No. 6,194,551). Alternatively or additionally, cysteine residue(s) may be introduced in the Fc region, thereby allowing interchain disulfide bond formation in this region. The homodimeric antibody thus generated may have improved or reduced internalization capability and/or increased or decreased complement-mediated cell killing. See Caron et al., J. Exp Med. 176:1191-1195 (1992) and Shopes, B. J. Immunol. 148:2918-2922 (1992), WO99/51642, Duncan & Winter Nature 322: 738-40 (1988); U.S. Pat. No. 5,648,260; U.S. Pat. No. 5,624,821; and W094/29351.

Methods of treatment

Some aspects of this disclosure provide methods for treating cancer, e.g., head and neck cancer, in a subject. Typically, such methods include administering an effective amount of a therapeutic composition comprising an ALK1 antagonist or inhibitor to a subject in need thereof, e.g., to a subject having head and neck cancer. In some embodiments, the composition includes a second therapeutic agent, e.g., a chemotherapeutic platinum agent. For example, in some embodiments, a method for treating a head and neck cancer in a subject is provided that includes administering an ALK1 antagonist and a chemotherapeutic platinum agent to the subject in an effective amount to treat the head and neck cancer.

As used herein, the terms “treatment,” “treat,” and “treating” refer to a clinical intervention aimed to reverse, alleviate, delay the onset of, or inhibit the progress of a disease or disorder, e.g., of head and neck cancer, or one or more symptoms thereof, as described herein. For example, treatment may result in complete or partial regression of a head and neck tumor, in a delay in tumor growth or progression, or in an alleviation of one or more symptoms associated with or caused by head and neck cancer. In some embodiments, treatment may be administered after one or more symptoms have developed and/or after a disease has been diagnosed. In other embodiments, treatment may be administered in the absence of symptoms. For example, treatment may be administered to a subject prior to the onset of symptoms (e.g., in light of a history of symptoms and/or in light of genetic or other susceptibility factors). Treatment may also be continued after symptoms have resolved, for example, after surgical removal of a head and neck cancer tumor, e.g., to prevent or delay the recurrence of the disease or of associated symptoms, e.g., the re-emergence of a tumor. In some embodiments, the disease or disorder being treated is associated with aberrant angiogenesis, e.g., pathological angiogenesis associated with a cancer or a tumor. In some embodiments, the disease is head and neck cancer. In some embodiments, the caner is positive for HPV.

The terms “effective amount” and “therapeutically effective amount,” as used herein, refer to the amount or concentration of an inventive compound, that, when administered to a subject, is effective to at least partially treat a condition from which the subject is suffering. In some embodiments, an effective amount of an ALK1 inhibitor is an amount the administration of which results in inhibition of at least about 50%, at least about 60%, at least about 70%, at least about 75%, at least about 80%, at least about 90%, at least about 95%, at least about 98%, at least about 99%, at least about 99.5%, or about 100% of ALK1 activity, angiogenesis, or tumor growth or progression as compared to a baseline level, for example, a level of ALK1 activity, angiogenesis, or tumor growth or progression in the absence of the inhibitor.

In some embodiments, the method comprises administering an ALK1 inhibitor that comprises an ALK1-ECD polypeptide. In some embodiments, the amino acid sequence of the ALK1-ECD polypeptide is at least 33%, 50%, 60%, 70%, 75%, 80%, 85%, 90%, 91%, 92%, 93%, 94%, 95%, 96%, 97%, 98%, or 99% identical to the sequence of amino acids 22-118 of SEQ ID NO: 1. In some embodiments, the amino acid sequence of the ALK1-ECD polypeptide is at least 97% identical to the sequence of amino acids 22-118 of SEQ ID NO: 1. In some embodiments, the C-terminal amino acid residue of the ALK1-ECD polypeptide is not glutamine 118 (Q118) of SEQ ID NO: 1. In some embodiments, the C-terminal amino acid residue of the ALK1-ECD polypeptide is not a glutamine residue. In some embodiments, the C-terminal amino acid residue of the ALK1-ECD polypeptide is proline 113 (P113), glycine 114 (G114), threonine 115 (T115), aspartic acid 116 (D116), glycine 117 (G117), leucine 119 (L119), alanine 120 (A120), leucine 121 (L121), isoleucine 122 (I122), or leucine 123 (L123) of SEQ ID NO: 1. In some embodiments, the C-terminal amino acid residue of the ALK1-ECD polypeptide is any of residues 110-130 of SEQ ID NO: 1.

In some embodiments, the ALK1-ECD polypeptide is fused to an Fc portion of an immunoglobulin thus forming an ALK1-Fc fusion protein. In some embodiments, the Fc portion is an Fc portion of a human IgG1. In some embodiments, the amino acid sequence of the ALK1-Fc fusion protein comprises the sequence of SEQ ID NO: 3 or SEQ ID NO: 4. In some embodiments, the amino acid sequence of the ALK1-Fc fusion protein is at least 90% identical to the amino acid sequence of SEQ ID NO: 3 OR SEQ ID NO: 4. In some embodiments, the amino acid sequence of the ALK1-Fc fusion protein is at least 80%, 85%, 90%, 91%, 92%, 93%, 94%, 95%, 96%, 97%, 98%, or 99% identical to the amino acid sequence of SEQ ID NO: 3 or SEQ ID NO: 4. In some embodiments, at least 80%, 85%, 90%, 91%, 92%, 93%, 94%, 95%, 96%, 97%, 98%, or 99% of the ALK1-Fc fusion protein is in a dimeric form. In some embodiments, 100% of the ALK1-Fc fusion protein is in a dimeric form.

In some embodiments, the ALK1-ECD polypeptide and/or the ALK1-Fc fusion protein is glycosylated. In some embodiments, the ALK1-ECD polypeptide and/or the ALK1-Fc fusion protein binds BMP9 or BMP10 with a K_{D} of less than 1×10⁻⁷M, less than 1×10⁻⁸M, less than 1×10⁻⁹M, less than 1×10⁻¹⁰M, less than 1×10⁻¹¹M, or less than 1×10⁻¹²M. In some embodiments, the ALK1-ECD polypeptide and/or the ALK1-Fc fusion protein does not show significant or substantial binding to TGFβ-1, e.g., in that it binds to TGFβ-1 with a K_{D} of greater than 1×10⁻⁶M.

In some embodiments, the ALK1 antagonist is an antibody that interferes with ALK1 binding BMP9 and/or BMP10. In some embodiments, the antibody binds to an ALK1-ECD polypeptide with a K_{D} of less than 5×10⁻⁸M, less than 1×10⁻⁹ M, less than 1×10⁻¹⁰ M, less than 1×10⁻¹¹ M, or less than 1×10⁻¹² M. In other embodiments, the antibody binds to an ALK1 ligand, e.g., to BMP9 or BMP10 with a K_{D} of less than 5×10⁻⁸ M, less than 1×10⁻⁹ M, less than 1×10⁻¹⁰ M, less than 1×10⁻¹¹ M, or less than 1×10⁻¹² M. In some embodiments, the method of treatment includes administering a plurality of antibodies against two or more ALK1 ligands, e.g., an antibody against BMP9 and an antibody against BMP10. The antibody (or antibodies) is (are) administered in an amount effective to inhibit angiogenesis stimulated by an ALK1 ligand in the subject.

In some embodiments, the ALK1 antagonist is administered together with a chemotherapeutic platinum agent. In some embodiments, the chemotherapeutic platinum agent comprises a coordination complex of platinum. In some embodiments, the chemotherapeutic platinum agent is cisplatin, carboplatin, oxaliplatin, satraplatin, picoplatin, Nedaplatin, Triplatin, Lipoplatin, or any combination thereof. Additional suitable chemotherapeutic platinum agents will be apparent to those of skill in the art based on the instant disclosure.

One advantage of using a combination of an ALK1 inhibitor and a chemotherapeutic platinum agent is that the therapeutic effect of the combination is typically improved as compared to the effect that can be achieved by using the maximum tolerated dose or the maximum effective dose of either component alone. For example, chemotherapeutic platinum agents are typically tolerated to a maximum dose, above which the toxicity of the respective drug can result in serious side effects. To give one example, the maximum tolerated drug for cisplatin, a commonly used chemotherapeutic platinum agent, is typically about 5 mg/kg/day for most patients. It will be understood that the maximum tolerated dose will vary for each subject, and depend on factors such as overall health, age, gender, and specific metabolic conditions and intolerances. Treatment with doses of chemotherapeutic platinum agents above the maximum tolerated dose is typically not feasible because of the associated severe risks to the health of the patient. However, a combination of an ALK1 inhibitor, e.g., an ALK1-Fc fusion protein such as Dalantercept, with a chemotherapeutic platinum agent, e.g., cisplatin, can be employed to achieve an improved therapeutic effect without the problem of increased toxicity.

For example, in some embodiments, the ALK1 inhibitor (e.g., the ALK1-ECD polypeptide and/or the ALK1-Fc fusion protein) is administered to the subject at a dosage of 0.1-30 mg/kg/day, while the chemotherapeutic platinum agent is administered at a dosage of 0.1-10 mg/kg/day. In some embodiments, the chemotherapeutic platinum agent is administered at the maximum tolerated dose, e.g., at 0.1 mg/kg/day, 0.2 mg/kg/day, 0.3 mg/kg/day, 0.4 mg/kg/day, 0.5 mg/kg/day, 0.6 mg/kg/day, 0.7 mg/kg/day, 0.8 mg/kg/day, 0.9 mg/kg/day, 1 mg/kg/day, 1.5 mg/kg/day, 2 mg/kg/day, 2.5 mg/kg/day, 3 mg/kg/day, 3.5 mg/kg/day, 4 mg/kg/day, 4.5 mg/kg/day, 5 mg/kg/day, 5.5 mg/kg/day, 6 mg/kg/day, 6.5 mg/kg/day, 7 mg/kg/day, 7.5 mg/kg/day, 8 mg/kg/day, 8.5 mg/kg/day, 9 mg/kg/day, 9.5 mg/kg/day, or 10 mg/kg/day, and the ALK1 inhibitor is administered at a dosage of 0.1-30 mg/kg/day. In some embodiments, the ALK1 inhibitor is administered to the subject at a dosage of 0.1-30 mg/kg/day, e.g., at a dosage of 0.1 mg/kg/day, 0.2 mg/kg/day, 0.3 mg/kg/day, 0.4 mg/kg/day, 0.5 mg/kg/day, 0.6 mg/kg/day, 0.7 mg/kg/day, 0.8 mg/kg/day, 0.9 mg/kg/day, 1 mg/kg/day, 2 mg/kg/day, 2.5 mg/kg/day, 3 mg/kg/day, 4 mg/kg/day, 5 mg/kg/day, 6 mg/kg/day, 7 mg/kg/day, 8 mg/kg/day, 9 mg/kg/day, 10 mg/kg/day, 11 mg/kg/day, 12 mg/kg/day, 12.5 mg/kg/day, 13 mg/kg/day, 14 mg/kg/day, 15 mg/kg/day, 16 mg/kg/day, 17 mg/kg/day, 17.5 mg/kg/day, 18 mg/kg/day, 19 mg/kg/day, 20 mg/kg/day, 21 mg/kg/day, 22 mg/kg/day, 23 mg/kg/day, 24 mg/kg/day, 25 mg/kg/day, 26 mg/kg/day, 27 mg/kg/day, 28 mg/kg/day, 29 mg/kg/day, or 30 mg/kg/day. In some embodiments, the chemotherapeutic platinum agent is administered to the subject at a dosage of 0.1-5 mg/kg/day. In some embodiments, the chemotherapeutic platinum agent is administered to the subject at a dosage of 0.1 mg/kg/day, 0.2 mg/kg/day, 0.3 mg/kg/day, 0.4 mg/kg/day, 0.5 mg/kg/day, 0.6 mg/kg/day, 0.7 mg/kg/day, 0.8 mg/kg/day, 0.9 mg/kg/day, 1.0 mg/kg/day, 1.5 mg/kg/day, 2.0 mg/kg/day, 2.5 mg/kg/day, 3.0 mg/kg/day, 3.5 mg/kg/day, 4.0 mg/kg/day, 4.5 mg/kg/day, 5 mg/kg/day, 5.5 mg/kg/day, 6 mg/kg/day, 6.5 mg/kg/day, 7 mg/kg/day, 7.5 mg/kg/day, 8 mg/kg/day, 8.5 mg/kg/day, 9 mg/kg/day, 9.5 mg/kg/day, or 10 mg/kg/day. In some embodiments, the ratio of the ALK1 inhibitor and the chemotherapeutic platinum agent is 10:1-1:10 (weight:weight), for example, 1:2, 2:1, 1: 2.5, 2:5:1, 1:3, 3:1, 1:4, 4:1, 1:5, 5:1, 1:6, 6:1, 1:7, 7:1, 1:8, 8:1, 1:9, 9:1, 1:10, or 10:1. In some embodiments, the ALK1-ECD polypeptide and/or the ALK1-Fc fusion protein is administered to the subject at a dose of 10 mg/kg/day, and the chemotherapeutic platinum agent is administered to the subject at a dose of 5 mg/kg/day.

In some embodiments, the daily dosage of the ALK1 inhibitor and/or the chemotherapeutic platinum agent is administered to the subject in a single dose, while in other embodiments, the daily dosage is administered to the subject in two or more doses (e.g., in the morning and in the evening; in the morning, midday, and evening; every hour; every 2, 3, 4, 5, 6, 7, 8, or 12 hours, or in other intervals). In some embodiments, the dosage for multiple days is administered to the subject in a single dose, e.g., in a controlled-release formulation.

Dosages provided herein are exemplary and are not meant to be limiting. For example, in some embodiments, dosages that are above 5-10 mg/kg/day of the chemotherapeutic platinum agent and/or dosages that are above 10-30 mg/kg/day of the ALK1 inhibitor are envisioned. In some embodiments, the respective drug or compound is administered at or below the maximum tolerated dose, which, in some embodiments, may be the maximum dose that can be administered to the patient without an unreasonable risk of severe side effects or of side effects that outweigh the clinical benefit of administering the drug or compound. The maximum tolerated dose will depend, inter alia, on the specific compound used and the health status of the patient. Typical maximum tolerated doses are known to those of skill in the art. For example, typical maximum tolerated doses may be, in some embodiments, 60-100 mg/m² body surface for cisplatin; 0.9-1.1 mg/m ² for Triplatin; and 600-800 mg/m² for Carboplatin. Those of skill in the art will be aware of suitable methods to convert dosages provided in mg/m² into dosages of mg/kg/day for a given patient, e.g., according to methods well-known in the art, which include, for example, those described in DuBois et al., A formula to estimate the approximate surface area if height and weight be known. Arch Int Med 1916;17:863-71; Gehan et al., Estimation of human body surface area from height and weight. Cancer Chemother Rep 1970;54:225-35; Haycock et al., Geometric method for measuring body surface area: A height-weight formula validated in infants, children and adults. J Pediatr 1978;93:62-6; and Mosteller et al., Simplified calculation of body-surface area. N Engl J Med 1987;317:1098; the entire contents of each of which are incorporated herein by reference

In some embodiments, the method comprises treating a cancer in a subject, e.g., by administering an ALK1 inhibitor, either alone or in combination with another therapeutic agent, to a subject having a cancer, e.g., head and neck cancer, multiple myeloma, melanoma, lung cancer, pancreatic cancer (e.g., tumors of the pancreatic endocrine tissue), or breast cancer (e.g., primary breast cancer or metastatic breast cancer; Estrogen receptor positive (ER+) or estrogen receptor negative (ER+). In some embodiment, the cancer is head and neck cancer.

The term “head and neck cancer,” as used herein refers to a group of biologically similar malignant proliferative diseases that are characterized by tumors of the head and neck, e.g., tumors in the lip, oral cavity (mouth), nasal cavity (inside the nose), paranasal sinuses, pharynx, and larynx. In the majority of cases, head and neck cancers are squamous cell carcinomas (SCCHN), originating from the mucosal lining (epithelium) of these regions. Head and neck cancers often spread to the lymph nodes of the neck, which is often the first (and sometimes only) sign of the disease at the time of diagnosis. Head and neck cancer is associated with environmental and lifestyle risk factors, including tobacco smoking, alcohol consumption, UV light, particular chemicals, and certain strains of viruses, e.g., HPV. Head and neck cancers often have a poor prognosis, are frequently aggressive in their biologic behavior; and patients with head and neck cancer are at a high risk of developing a recurrent tumor even after an initial tumor is surgically removed or otherwise treated.

Some aspects of this disclosure provide methods for identifying a cancer as responsive to treatment with an ALK1 inhibitor, either alone or in combination with another therapeutic agent, e.g., a chemotherapeutic platinum agent or a VEGF antagonist. In some embodiment, this identification is based on the cancer being positive for HPV. Some aspects of this disclosure provide methods for selecting a subject having a cancer for treatment with an ALK1 inhibitor, either alone or in combination with another therapeutic agent, e.g., a chemotherapeutic platinum agent or a VEGF antagonist selecting a cancer for treatment, based on the cancer being HPV positive. Some aspects of this disclosure provide methods for administering an ALK1 inhibitor, either alone or in combination with another therapeutic agent, e.g., a chemotherapeutic platinum agent or a VEGF antagonist, to a subject having a cancer, e.g., a head and neck cancer, based on the cancer being HPV positive.

Suitable methods for the detection of HPV in a subject include detecting viral DNA or proteins according to well-established methods. For example, HPV DNA may be detected in a biopsy taken from a head and neck cancer tumor via PCR methods using appropriate primers.

HPV testing may also be done according to the 2013 NCCN guidelines for cancers of the oropharynx. For example, by either immunohistochemistry for analysis of p16 expression or HPV in situ hybridization for detection of HPV DNA in tumor cell nuclei. See NCCN Clinical Practice Guidelines in Oncology (NCCN Guidelines)—Head and Neck Cancer, Version 2.2013, May 29, 2013, Natural Comprehensive Cancer Network, Inc., the entire contents of which are incorporated herein by reference.

HPV DNA may also be detected in the serum of a subject, which may be preferable in some embodiments, as it avoids the need for a tumor biopsy. Suitable detection methods and reagents (e.g., PCR primers) for the detection of HPV in a subject include, but are not limited to, those described in Molijn et al., Molecular diagnosis of human papillomavirus (HPV) infections, Journal of Clinical Virology 32S (2005) S43-S51; and Capone et al., Detection and Quantitation of Human Papillomavirus (HPV) DNA in the Sera of Patients with HPV-associated Head and Neck Squamous Cell Carcinoma Clin Cancer Res November 2000 6; 417; the entire contents of each of which are incorporated herein by reference. Additional suitable methods and reagents will be apparent to those of skill in the art based on the instant disclosure.

Some aspects of this disclosure provide methods for treating HPV-positive head and neck cancer in a subject. In some embodiments, the method comprises administering to a subject in need thereof an agent selected from the group consisting of (i) an ALK1-extracellular domain (ALK1-ECD) polypeptide; (ii) an antibody that binds to an ALK1 polypeptide comprising amino acids 22-118 of SEQ ID NO: 1; and (iii) an antibody that binds to BMP9 or BMP10; in an amount sufficient to treat the head and neck cancer in the subject. In some embodiments, the amino acid sequence of the ALK1-ECD polypeptide is at least 90% identical to the sequence of amino acids 22-118 of SEQ ID NO: 1. In some embodiments, the amino acid sequence of the ALK1-ECD polypeptide is at least 33%, 50%, 60%, 70%, 75%, 80%, 85%, 90%, 91%, 92%, 93%, 94%, 95%, 96%, 97%, 98%, or 99% identical to the sequence of amino acids 22-118 of SEQ ID NO: 1. In some embodiments, the C-terminal amino acid residue of the ALK1-ECD polypeptide is not glutamine 118 (Q118) of SEQ ID NO: 1. In some embodiments, the C-terminal amino acid residue of the ALK1-ECD polypeptide is not a glutamine residue. In some embodiments, the C-terminal amino acid residue of the ALK1-ECD polypeptide is proline 113 (P113), glycine 114 (G114), threonine 115 (T115), aspartic acid 116 (D116), glycine 117 (G117), leucine 119 (L119), alanine 120 (A120), leucine 121 (L121), isoleucine 122 (I122), or leucine 123 (L123) of SEQ ID NO: 1.

In some embodiments, the ALK1-ECD polypeptide is fused to an Fc portion of an immunoglobulin thus forming an ALK1-Fc fusion protein. In some embodiments, the Fc portion is an Fc portion of a human IgG1. In some embodiments, the amino acid sequence of the ALK1-Fc fusion protein comprises the sequence of SEQ ID NO: 3 or SEQ ID NO: 4. In some embodiments, the amino acid sequence of the ALK1-Fc fusion protein is at least 33%, 50%, 60%, 70%, 75%, 80%, 85%, 90%, 91%, 92%, 93%, 94%, 95%, 96%, 97%, 98%, or 99% identical to the amino acid sequence of SEQ ID NO: 3 or SEQ ID NO: 4. In some embodiments, the amino acid sequence of the ALK1-Fc fusion protein is at least 33%, 50%, 60%, 70%, 75%, 80%, 85%, 90%, 91%, 92%, 93%, 94%, 95%, 96%, 97%, 98%, or 99% identical to the amino acid sequence of SEQ ID NO: 3 or SEQ ID NO: 4. In some embodiments, at least 90% of the ALK1-Fc fusion protein is in a dimeric form. In some embodiments, the ALK1-ECD polypeptide and/or the ALK1-Fc fusion protein is glycosylated. In some embodiments, the ALK1-ECD polypeptide and/or the ALK1-Fc fusion protein binds BMP9 or BMP10 with a K_{D} of less than 1×10⁻⁷M, e.g., with a K_{D} of less than 1×10⁻⁸M, less than 1×10⁻⁹M, less than 1×10⁻¹⁰M, or less than 1×10⁻¹¹M. In some embodiments, the ALK1-ECD polypeptide and/or the ALK1-Fc fusion protein does not exhibit substantial binding to TGFβ-1, e.g., in that it binds to TGFβ-1 with a K_{D} of greater than 1×10⁻⁶M. In some embodiments, the ALK1-ECD polypeptide and/or the ALK1-Fc fusion protein is administered in a pharmaceutical preparation.

In some embodiments, the antibody of (ii) binds to an ALK1-ECD polypeptide with a K_{D} of less than 5×10⁻⁸M. In some embodiments, the antibody of (ii) binds to an ALK1-ECD polypeptide with a K_{D} of less than 1×10⁻¹⁰ M. In some embodiments, the antibody of (ii) inhibits angiogenesis stimulated by an ALK1 ligand. In some embodiments, the antibody of (ii) inhibits the binding of BMP9 or BMP10 to an ALK1-ECD polypeptide. In some embodiments, the antibody of (iii) binds to an ALK1-ECD polypeptide with a K_{D} of less than 5×10⁻⁸ M. In some embodiments, the antibody of (iii) inhibits angiogenesis stimulated by at least one ALK1 ligand.

In some embodiments, the ALK1-ECD polypeptide and/or the ALK1-Fc fusion protein is administered to the subject at a dosage of 0.1-30 mg/kg/day. In some embodiments, the ALK1-ECD polypeptide and/or the ALK1-Fc fusion protein is administered to the subject at a dosage of 0.1, 0.2, 0.3, 0.4, 0.5, 0.6, 0.7, 0.8, 0.9, 1, 2, 3, 4, 5, 10, 15, 20, 25, or 30 mg/kg/day.

In some embodiments, the method further comprises administering a chemotherapeutic platinum agent to the subject. In some embodiments, the chemotherapeutic platinum agent comprises a coordination complex of platinum. In some embodiments, the chemotherapeutic platinum agent is cisplatin, carboplatin, oxaliplatin, satraplatin, picoplatin, Nedaplatin, Triplatin, Lipoplatin, or any combination thereof.

In some embodiments, the chemotherapeutic platinum agent is administered to the subject at a dosage of 0.1-10 mg/kg/day. In some embodiments, the chemotherapeutic platinum agent is administered to the subject at a dosage of 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, or 10 mg/kg/day. In some embodiments, the ALK1-ECD polypeptide and/or the ALK1-Fc fusion protein is administered to the subject at a dose of 10 mg/kg/day, and the chemotherapeutic platinum agent is administered to the subject at a dose of 5 mg/kg/day.

For example, in some embodiments, the ALK1 inhibitor (e.g., the ALK1-ECD polypeptide and/or the ALK1-Fc fusion protein) is administered to the subject at a dosage of 0.1-30 mg/kg/day, while the chemotherapeutic platinum agent is administered at a dosage of 0.5-5 mg/kg/day. In some embodiments, the chemotherapeutic platinum agent is administered at the maximum tolerated dose, e.g., at 0.1 mg/kg/day, 0.2 mg/kg/day, 0.3 mg/kg/day, 0.4 mg/kg/day, 0.5 mg/kg/day, 0.6 mg/kg/day, 0.7 mg/kg/day, 0.8 mg/kg/day, 0.9 mg/kg/day, 1 mg/kg/day, 1.5 mg/kg/day, 2 mg/kg/day, 2.5 mg/kg/day, 3 mg/kg/day, 3.5 mg/kg/day, 4 mg/kg/day, 4.5 mg/kg/day, 5 mg/kg/day, 5.5 mg/kg/day, 6 mg/kg/day, 6.5 mg/kg/day, 7 mg/kg/day, 7.5 mg/kg/day, 8 mg/kg/day, 8.5 mg/kg/day, 9 mg/kg/day, 9.5 mg/kg/day, or 10 mg/kg/day, and the ALK1 inhibitor is administered at a dosage of 0.1-30 mg/kg/day. In some embodiments, the ALK1 inhibitor is administered to the subject at a dosage of 0.1-30 mg/kg/day, e.g., at a dosage of 0.1 mg/kg/day, 0.2 mg/kg/day, 0.3 mg/kg/day, 0.4 mg/kg/day, 0.5 mg/kg/day, 0.6 mg/kg/day, 0.7 mg/kg/day, 0.8 mg/kg/day, 0.9 mg/kg/day, 1 mg/kg/day, 2 mg/kg/day, 2.5 mg/kg/day, 3 mg/kg/day, 4 mg/kg/day, 5 mg/kg/day, 6 mg/kg/day, 7 mg/kg/day, 8 mg/kg/day, 9 mg/kg/day, 10 mg/kg/day, 11 mg/kg/day, 12 mg/kg/day, 12.5 mg/kg/day, 13 mg/kg/day, 14 mg/kg/day, 15 mg/kg/day, 16 mg/kg/day, 17 mg/kg/day, 17.5 mg/kg/day, 18 mg/kg/day, 19 mg/kg/day, 20 mg/kg/day, 21 mg/kg/day, 22 mg/kg/day, 23 mg/kg/day, 24 mg/kg/day, 25 mg/kg/day, 26 mg/kg/day, 27 mg/kg/day, 28 mg/kg/day, 29 mg/kg/day, or 30 mg/kg/day. In some embodiments, the chemotherapeutic platinum agent is administered to the subject at a dosage of 0.1-5 mg/kg/day. In some embodiments, the chemotherapeutic platinum agent is administered to the subject at a dosage of 0.1 mg/kg/day, 0.2 mg/kg/day, 0.3 mg/kg/day, 0.4 mg/kg/day, 0.5 mg/kg/day, 0.6 mg/kg/day, 0.7 mg/kg/day, 0.8 mg/kg/day, 0.9 mg/kg/day, 1 mg/kg/day, 1.5 mg/kg/day, 2 mg/kg/day, 2.5 mg/kg/day, 3 mg/kg/day, 3.5 mg/kg/day, 4 mg/kg/day, 4.5 mg/kg/day, 5 mg/kg/day, 5.5 mg/kg/day, 6 mg/kg/day, 6.5 mg/kg/day, 7 mg/kg/day, 7.5 mg/kg/day, 8 mg/kg/day, 8.5 mg/kg/day, 9 mg/kg/day, 9.5 mg/kg/day, or 10 mg/kg/day. In some embodiments, the ratio of the ALK1 inhibitor and the chemotherapeutic platinum agent is 10:1-1:10 (weight:weight), for example, 1:2, 2:1, 1: 2.5, 2:5:1, 1:3, 3:1, 1:4, 4:1, 1:5, 5:1, 1:6, 6:1, 1:7, 7:1, 1:8, 8:1, 1:9, 9:1, 1:10, or 10:1. In some embodiments, the ALK1-ECD polypeptide and/or the ALK1-Fc fusion protein is administered to the subject at a dose of 10 mg/kg/day, and the chemotherapeutic platinum agent is administered to the subject at a dose of 5 mg/kg/day.

In some embodiments, the head and neck cancer is a recurrent or metastatic squamous cell carcinoma. In some embodiments, the method further comprises determining that the head and neck cancer is HPV-positive. In some embodiments, the method further comprises identifying the head and neck cancer as responsive to treatment with the agent based on the head and neck cancer being positive for HPV. In some embodiments, the agent are administered based on the head and neck cancer being positive for HPV.

Some aspects of this disclosure provide methods for identifying whether a cancer responds to treatment with an ALK1 antagonist. In some embodiments, the method comprises (a) determining whether a head and neck cancer in a subject is HPV positive; wherein, if the head and neck cancer is positive for HPV, then the cancer is identified to respond to treatment with an ALK1 antagonist. In some embodiments, the ALK1 antagonist is an agent selected from the group consisting of (i) an ALK1-extracellular domain (ALK1-ECD) polypeptide; (ii) an antibody that binds to an ALK1 polypeptide comprising amino acids 22-118 of SEQ ID NO: 1; and (iii) an antibody that binds to BMP9 or BMP10. In some embodiments, the method further comprises (b) administering the ALK1 antagonist to the subject in an amount effective to treat the head and neck cancer. In some embodiments, the method further comprises obtaining a biopsy from the head and neck cancer in the subject.

In some embodiments, the amino acid sequence of the ALK1-ECD polypeptide is at least 90% identical to the sequence of amino acids 22-118 of SEQ ID NO: 1. In some embodiments, the amino acid sequence of the ALK1-ECD polypeptide is at least 97% identical to the sequence of amino acids 22-118 of SEQ ID NO: 1. In some embodiments, the C-terminal amino acid residue of the ALK1-ECD polypeptide is not glutamine 118 (Q118) of SEQ ID NO: 1. In some embodiments, the C-terminal amino acid residue of the ALK1-ECD polypeptide is not a glutamine residue. In some embodiments, the C-terminal amino acid residue of the ALK1-ECD polypeptide is proline 113 (P113), glycine 114 (G114), threonine 115 (T115), aspartic acid 116 (D116), glycine 117 (G117), leucine 119 (L119), alanine 120 (Al20), leucine 121 (L121), isoleucine 122 (1122), or leucine 123 (L123) of SEQ ID NO: 1. In some embodiments, the ALK1-ECD polypeptide is fused to an Fc portion of an immunoglobulin thus forming an ALK1-Fc fusion protein. In some embodiments, the Fc portion is an Fc portion of a human IgG1. In some embodiments, the amino acid sequence of the ALK1-Fc fusion protein comprises the sequence of SEQ ID NO: 3 or SEQ ID NO: 4. In some embodiments, the amino acid sequence of the ALK1-Fc fusion protein is at least 90% identical to the amino acid sequence of SEQ ID NO: 3 or SEQ ID NO: 4. In some embodiments, the amino acid sequence of the ALK1-Fc fusion protein is at least 97% identical to the amino acid sequence of SEQ ID NO: 3 or SEQ ID NO: 4. In some embodiments, at least 90% of the ALK1-Fc fusion protein is in a dimeric form. In some embodiments, the ALK1-ECD polypeptide and/or the ALK1-Fc fusion protein is glycosylated. In some embodiments, the ALK1-Fc fusion protein is Dalantercept. In some embodiments, the ALK1-ECD polypeptide and/or the ALK1-Fc fusion protein is administered to the subject at a dosage of 0.1-30 mg/kg/day. In some embodiments, the head and neck cancer is a recurrent or metastatic squamous cell carcinoma.

Some aspects of this disclosure provide methods that comprise (a) obtaining a biopsy from a head and neck cancer in a subject; (b) determining whether the head and neck cancer is HPV positive; and (c) if the head and neck cancer is positive for HPV, identifying the head and neck cancer as responsive to treatment with an agent selected from the group consisting of (i) an ALK1-extracellular domain (ALK1-ECD) polypeptide; (ii) an antibody that binds to an ALK1 polypeptide comprising amino acids 22-118 of SEQ ID NO: 1; and (iii) an antibody that binds to BMP9 or BMP10. In some embodiments, the method further comprises administering the agent to the subject in an amount effective to treat the head and neck cancer.

In some embodiments, the amino acid sequence of the ALK1-ECD polypeptide is at least 90% identical to the sequence of amino acids 22-118 of SEQ ID NO: 1. In some embodiments, the amino acid sequence of the ALK1-ECD polypeptide is at least 97% identical to the sequence of amino acids 22-118 of SEQ ID NO: 1. In some embodiments, the C-terminal amino acid residue of the ALK1-ECD polypeptide is not glutamine 118 (Q118) of SEQ ID NO: 1. In some embodiments, the C-terminal amino acid residue of the ALK1-ECD polypeptide is not a glutamine residue. In some embodiments, the C-terminal amino acid residue of the ALK1-ECD polypeptide is proline 113 (P113), glycine 114 (G114), threonine 115 (T115), aspartic acid 116 (D116), glycine 117 (G117), leucine 119 (L119), alanine 120 (Al20), leucine 121 (L121), isoleucine 122 (I122), or leucine 123 (L123) of SEQ ID NO: 1. In some embodiments, the ALK1-ECD polypeptide is fused to an Fc portion of an immunoglobulin thus forming an ALK1-Fc fusion protein. In some embodiments, the Fc portion is an Fc portion of a human IgG1. In some embodiments, the amino acid sequence of the ALK1-Fc fusion protein comprises the sequence of SEQ ID NO: 3 or SEQ ID NO: 4. In some embodiments, the amino acid sequence of the ALK1-Fc fusion protein is at least 90% identical to the amino acid sequence of SEQ ID NO: 3 or SEQ ID NO: 4. In some embodiments, the amino acid sequence of the ALK1-Fc fusion protein is at least 97% identical to the amino acid sequence of SEQ ID NO: 3 or SEQ ID NO: 4. In some embodiments, at least 90% of the ALK1-Fc fusion protein is in a dimeric form. In some embodiments, the ALK1-ECD polypeptide and/or the ALK1-Fc fusion protein is glycosylated. In some embodiments, the ALK1-Fc fusion protein is Dalantercept. In some embodiments, the ALK1-ECD polypeptide is administered to the subject at a dosage of 0.1-30 mg/kg/day. In some embodiments, the head and neck cancer is a recurrent or metastatic squamous cell carcinoma.

In general, one or more therapeutic agents can be administered in any of the therapeutic methods provided herein. The methods of the disclosure also include co-administration with other medicaments that are used to treat the respective condition, e.g., cancers such as head and neck cancer. When administering more than one agent or a combination of agents and medicaments, e.g., a combination of an ALK1 inhibitor and a chemotherapeutic platinum agent, administration can occur simultaneously or sequentially in time. The therapeutic agents and/or medicaments may be administered by different routes of administration or by the same route of administration. If administered via the same route, the agents may be administered through the same point of entry, e.g., the same intravenous port, or through different points of entry.

### Pharmaceutical Compositions

Some aspects of this disclosure provide pharmaceutical compositions for the treatment of head and neck cancer in a subject. In some embodiments, the composition comprises (a) an agent selected from the group consisting of (i) an ALK1-extracellular domain (ALK1-ECD) polypeptide; (ii) an antibody that binds to BMP9 or BMP10; or (iii) an antibody that binds to an ALK1 polypeptide comprising amino acids 22-118 of SEQ ID NO: 1; and (b) a chemotherapeutic platinum agent, wherein the agent of (a) and the chemotherapeutic platinum agent are in an amount sufficient to treat a head and neck cancer in the subject. In some embodiments, the amino acid sequence of the ALK1-ECD polypeptide is at least 90% identical to the sequence of amino acids 22-118 of SEQ ID NO: 1. In some embodiments, the amino acid sequence of the ALK1-ECD polypeptide is at least 97% identical to the sequence of amino acids 22-118 of SEQ ID NO: 1. In some embodiments, the C-terminal amino acid residue of the ALK1-ECD polypeptide is not glutamine 118 (Q118) of SEQ ID NO: 1. In some embodiments, the C-terminal amino acid residue of the ALK1-ECD polypeptide is not a glutamine residue. In some embodiments, the C-terminal amino acid residue of the ALK1-ECD polypeptide is proline 113 (P113), glycine 114 (G114), threonine 115 (T115), aspartic acid 116 (D116), glycine 117 (G117), leucine 119 (L119), alanine 120 (A120), leucine 121 (L121), isoleucine 122 (I122), or leucine 123 (L123) of SEQ ID NO: 1. In some embodiments, the ALK1-ECD polypeptide is fused to an Fc portion of an immunoglobulin thus forming an ALK1-Fc fusion protein. In some embodiments, the Fc portion is an Fc portion of a human IgG1. In some embodiments, the ALK1-Fc fusion protein comprises the sequence of SEQ ID NO: 3 or SEQ ID NO: 4. In some embodiments, the amino acid sequence of the ALK1-Fc fusion protein is at least 90% identical to the amino acid sequence of SEQ ID NO: 3 or SEQ ID NO: 4. In some embodiments, the amino acid sequence of the ALK1-Fc fusion protein is at least 97% identical to the amino acid sequence of SEQ ID NO: 3 or SEQ ID NO: 4. In some embodiments, the ALK1-ECD polypeptide and/or the ALK1-Fc fusion protein binds BMP9 or BMP10 with a K_{D} of less than 1×10⁻⁷M. In some embodiments, the ALK1-ECD polypeptide and/or the ALK1-Fc fusion protein binds to TGFβ-1 with a K_{D} of greater than 1×10⁻⁶M. In some embodiments, the ALK1-ECD polypeptide is administered in a pharmaceutical preparation wherein at least 90% of the ALK1-Fc fusion protein is in a dimeric form.

In some embodiments, the antibody of (ii) binds to an ALK1-ECD polypeptide with a K_{D} of less than 5×10⁻⁸M. In some embodiments, the antibody of (ii) binds to an ALK1-ECD polypeptide with a K_{D} of less than 1×10⁻¹⁰ M. In some embodiments, the antibody of (ii) inhibits angiogenesis stimulated by an ALK1 ligand. In some embodiments, the antibody of (ii) inhibits the binding of BMP9 or BMP10 to an ALK1-ECD polypeptide. In some embodiments, the antibody of (iii) binds to an ALK1-ECD polypeptide with a K_{D} of less than 5×10⁻⁸ M. In some embodiments, the antibody of (iii) inhibits angiogenesis stimulated by at least one ALK1 ligand.

In some embodiments, the chemotherapeutic platinum agent comprises a coordination complexes of platinum. In some embodiments, the chemotherapeutic platinum agent is cisplatin, carboplatin, oxaliplatin, satraplatin, picoplatin, Nedaplatin, Triplatin, Lipoplatin, or any combination thereof. Some exemplary suitable platinum agents are provided herein and additional suitable platinum agents include those reported in Donzelli et al., (2004) Neurotoxicity of platinum compounds: Comparison of the effects of cisplatin and oxaliplatin on the human neuroblastoma cell line SH-SY5Y. Journal of neuro-oncology 67 (1-2): 65-73; Poklar et al., (1996) Influence of cisplatin intrastrand crosslinking on the conformation, thermal stability, and energetics of a 20-mer DNA duplex. Proc. Natl. Acad. Sci. U.S.A. 93 (15): 7606-11; Rudd et al., (1995) Persistence of cisplatin-induced DNA interstrand crosslinking in peripheral blood mononuclear cells from elderly and young individuals. Cancer Chemother. Pharmacol. 35 (4): 323-6; Cruet-Hennequart et al., (2008) Enhanced DNA-PK-mediated RPA2 hyperphosphorylation in DNA polymerase eta-deficient human cells treated with cisplatin and oxaliplatin. DNA Repair (Amst.) 7 (4): 582-96; Kelland (2007) The resurgence of platinum-based cancer chemotherapy. Nature Reviews Cancer 7 (8): 573-584; and Einhorn (1990) Treatment of testicular cancer: a new and improved model. J. Clin. Oncol. 8 (11): 1777-81; the entire contents of each of which are incorporated herein by reference.

The therapeutic agents described herein may be formulated into pharmaceutical compositions. Pharmaceutical compositions for use in accordance with the present disclosure may be formulated in conventional manner using one or more physiologically acceptable carriers or excipients. Such formulations will generally be substantially pyrogen free, in compliance with most regulatory requirements.

In certain embodiments, the therapeutic method of the disclosure includes administering the composition systemically, or locally as an implant or device. When administered, the therapeutic composition for use in this disclosure is in a pyrogen-free, physiologically acceptable form. Therapeutically useful agents other than the ALK1 antagonists which may also optionally be included in the composition as described above, e.g., chemotherapeutic platinum agents, may be administered simultaneously or sequentially with the subject compounds, e.g., ALK1 ECD polypeptides or any of the antibodies disclosed herein, in the methods disclosed herein.

Typically, protein therapeutic agents disclosed herein will be administered parenterally, e.g., intravenously or subcutaneously. Pharmaceutical compositions suitable for parenteral administration may comprise one or more ALK1 ECD polypeptide or other antibodies in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

In some embodiments, the antibodies and ALK1 ECD proteins disclosed herein are administered in a pharmaceutical formulation. In some embodiments, the pharmaceutical formulation is a sterile aqueous solution, preferable of suitable concentration for injection. Such formulations typically comprise one or more antibodies or ALK1 ECD proteins disclosed herein dissolved or suspended in a sterile pharmaceutically acceptable base, such as a buffered saline solution. Thimerosal, chlorobutanol, or other antimicrobial agents may also be included.

The disclosure provides formulations that may be varied to include acids and bases to adjust the pH; and buffering agents to keep the pH within a narrow range. Additional medicaments may be added to the formulation. These include, but are not limited to, chemotherapeutic platinum agents, pegaptanib, heparinase, ranibizumab, or glucocorticoids.

The compositions and formulations may, if desired, be presented in a pack or dispenser device which may contain one or more unit dosage forms containing the active ingredient. The pack may for example comprise metal or plastic foil, such as a blister pack. In some embodiments, the pack may contain one or more vials containing a powdered, freeze-dried, or lyophilized formulation of the pharmaceutical composition, e.g., a vial containing a lyophilized ALK1 inhibitor, such as, e.g., an ALK1-ECD polypeptide, an ALK1-Fc fusion protein, an ALK1-inhibitory antibody, or a combination of one or more ALK1 antagonists and a chemotherapeutic platinum agent, either in different vials or in the same vial. The pack or dispenser device may be accompanied by instructions for administration.

In some embodiments, a single dosage form, e.g., a single pill, container, vial, dispenser unit, or pack of the respective compound, e.g., of the ALK1 inhibitor or the chemotherapeutic platinum agent, contains 0.1-500 mg of the compound. For example, some embodiments of this disclosure provide a dosage form, e.g., a pill, container, vial, dispenser unit, or pack that comprises 0.1 mg, 0.2 mg, 0.3 mg, 0.4 mg, 0.5 mg, 0.6 mg, 0.7 mg, 0.8 mg, 0.9 mg, 1 mg, 1.5 mg, 2 mg, 2.5 mg, 3 mg, 3.5 mg, 4 mg, 4.5 mg, 5 mg, 5.5 mg, 6 mg, 6.5 mg, 7 mg, 7.5 mg, 8 mg, 8.5 mg, 9 mg, 9.5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 150 mg, 200 mg, 250 mg, 300 mg, 350 mg, 400 mg, 450 mg, or 500 mg of an ALK1 inhibitor, e.g., an ALK1-ECD polypeptide, an ALK1-Fc fusion protein, or an ALK1-inhibitory antibody and/or of a chemotherapeutic platinum agent.

It will be understood that where dosages are provided in mg/kg/day in this disclosure, the dosage refers to the dose given to an individual in mg per kg body weight of the individual per day of the treatment regimen. The skilled artisan will understand that the body weight used in such calculations is, in some embodiments, rounded to a full kg value, or to the closest kg, 2 kg, 2.5 kg, 5 kg, or 10 kg value. The disclosure of mg/kg values includes the disclosure of such values expressed in other unit systems, such as ounces, pounds, imperial units, metric units, natural units, or non-standard units. It will also be understood that a dosage of mg/kg/day may be effected by administering the respective amount once per day, by administering the respective fraction at multiple times per day (e.g., administering 50% of the daily dose twice per day, 33% three times a day, 25% four times a day, 20% five times a day, etc.), or administering a larger dose in less-than-daily intervals (e.g., administering twice the dose every other day, three times the dose every three days, four times the dose every four days, etc.). To illustrate, a dose of 5 mg/kg/day may be achieved, for example, by a single administration of 5 mg/kg once a day during the time of the treatment, by administrations of 1.25 mg/kg four times a day during the time of the treatment, by administration of 10 mg/kg every second day, or by administration of 35 mg/kg once a week. The particular dosage schedule will depend, inter alia, on the pharmacokinetic and toxicity characteristics of the administered drug,

It will be understood that dosages provided in mg/kg/day can be converted into dosages of mg/m² according to methods well-known in the art for an individual patient, including, for example, those described in DuBois et al.,A formula to estimate the approximate surface area if height and weight be known. Arch Int Med 1916;17:863-71; Gehan et al., Estimation of human body surface area from height and weight. Cancer Chemother Rep 1970;54:225-35; Haycock et al., Geometric method for measuring body surface area: A height-weight formula validated in infants, children and adults. J Pediatr 1978;93:62-6; and Mosteller et al., Simplified calculation of body-surface area. N Engl J Med 1987;317:1098; the entire contents of each of which are incorporated herein by reference.

Some of the embodiments, advantages, features, and uses of the technology disclosed herein will be more fully understood from the Examples below. The Examples are intended to illustrate some of the benefits of the present disclosure and to describe particular embodiments, but are not intended to exemplify the full scope of the disclosure and, accordingly, do not limit the scope of the disclosure.

### EXAMPLE

### Example 1

### Dalantercept, an ALK1/BMP9 Inhibitor of Angiogenesis, in Combination with Cisplatin Enhances Tumor Growth Inhibition in a Xenograft Model of Squamous Cell Carcinoma of the Head and Neck

Activin receptor-like kinase 1 (ALK1) is a key regulator of angiogenesis and vascular morphogenesis. ALK1 and its co-receptor endoglin are expressed on the surface of endothelial cells during active angiogenesis. Bone morphogenetic proteins (BMP) 9 and 10 are ligands that bind to ALK1 and induce activation of the heteromeric receptor complex, phosphorylation of SMAD1/5/8, and upregulation of specific genes involved in angiogenesis, such as Id-1 and TMEM100. BMP9 is overexpressed in the majority of squamous cell carcinoma of the head and neck (SCCHN). Dalantercept is an ALK1 extracellular domain-Fc fusion protein that selectively binds BMP9 and BMP10 with high affinity and antagonizes ALK1 signaling in vivo which results in defective vascular maturation and inhibition of tumor growth in preclinical models.

In a completed Phase 1 study in patients with advanced, refractory solid tumors dalantercept demonstrated signs of clinical activity in a variety of patients including patients with SCCHN who achieved an objective response or prolonged stable disease. A Phase 2 study with dalantercept in SCCHN is currently underway. Based upon the single agent activity of both dalantercept and cisplatin in SCCHN, we tested the feasibility of combining cisplatin with dalantercept in a mouse model of SCCHN. Combination treatment of dalantercept with cisplatin showed significant inhibition of tumor growth in RPMI2650 xenograft model (59% TGI on day 30) which was significantly better than either cisplatin (35% TGI, p=0.0077) or dalantercept (32% TGI, p=0.0002) monotherapy. No additional toxicity was observed in the combination treatment group compared to cisplatin monotherapy group. This data suggests that combination of dalantercept with cisplatin may result in enhanced clinical activity and justifies prospective evaluation in patients with SCCHN.

TABLE 1
Terms and abbreviations

| Term or abbreviation | Explanation |
|---|---|
| ALK1 | Activin receptor-like Kinase-1 |
| IACUC | Institutional Animal Care and Use Committee |
| kg | Kilograms |
| mg | Milligrams |
| Ml | Milliliters |
| mm | Millimeters |
| ACE-041, dalantercept | Soluble ALK1-IgG1 fusion protein |
| | (see, e.g., SEQ ID NO: 3 for amino acid |
| | sequence) |
| SC, SQ | Subcutaneous |
| SEM | Standard error of the mean |
| TBS | Modified Phosphate Buffered Saline |
| VEH | Vehicle |
| μl | Microliters |
| IV | Intravenous |
| IP | Intraperitoneal |
| QW | Once a week |
| TIW | Three times in a week |
| SFM | Serum free media |
| TGI | Tumor growth inhibition |
| GMDV | Geo mean of final tumor volume/ |
| | geo mean of initial tumor volume |

In Phase 1 trial with dalantercept several patients with squamous cell carcinoma of head and neck (SCCHN) showed stable disease. Cisplatin is one of the most commonly used standard of care treatment for this patient population. We wanted to test if combination of dalantercept with cisplatin was tolerated in a mouse tumor model of head and neck carcinoma and if the combination treatment would result in an additive or synergistic anti-tumor effect.

Test animals. Animal information is provided in Table 2.

TABLE 2
Test animal information

| | |
|---|---|
| Species and Strain | Mus musculus, (Hsd: Athymic Nude- |
| | Foxn1^{nu}) |
| Supplier | Harlan |
| Number of Animals Received | 60 females |
| Number Used on Study | 42 |
| Age at First Dose | Approximately 8 weeks |
| Actual Weight Range at First Dose | Min = 18, Max = 24 g |

Animals were acclimated to laboratory conditions for a minimum of 48 hours prior to the first dose. During this period all animals were observed for any signs of clinical abnormalities that would exclude them from study. Animals were assigned a study number on their cage cards and uniquely identified by ear notching.

The Institutional Animal Care and Use Committee (IACUC) of Acceleron Pharma approved all procedures related to this study design and found it to be in accordance with provisions of the USDA Animal Welfare Act, the PHS Policy on Humane Care and Use of Laboratory Animals, and the US Interagency Research Animal Committee Principles for the Utilization and Care of Research Animals.

Test animal housing and care. Animal husbandry was provided as described in Table 3.

TABLE 3
Animal Husbandry

| | |
|---|---|
| Feed | Teklad Diet 2020x |
| Water | Tap water via water bottles |
| Bedding | SaniChip ® certified hardwood bedding |
| Housing | Individually housed in polycarbonate cages suspended |
| | on stainless steel racks |
| Temperature | 18 to 29° C. |
| Range | |
| Humidity Range | 30 to 70% |
| Light Cycle | 12-hour light/12-hour dark, interrupted as necessary |
| | for study related events |
| Air Changes | Minimum of 10 air changes per hour |

Feed and water were provided ad libitum, unless otherwise noted. The bedding is routinely analyzed by the manufacturer for acceptable levels of heavy metals, aflatoxins, bacteria, yeasts, molds, and organophosphates prior to certification. No contaminants were known to be present in the feed, water, or bedding at levels that might have interfered with achieving the objectives of the study.

Environmental controls were set to maintain animal room conditions as shown in Table 3. Actual temperature and relative humidity in the animal room were monitored continuously by a computerized system. All environmental parameters were maintained within the protocol requirements, except if noted.

Vehicle control. Sterile filtered modified TBS (10 mM Tris, 137 mM NaCl, 2.7 mM KC1. pH=7.21). Storage: Stored at room temperature until use. Modified TBS was administered at 5 ml/kg by i.p. injection.

ACE-041, dalantercept (ALK1-IgG1). Lot #: PO7041-006xA. Diluted using sterile modified TBS to a concentration of 2.0 mg/ml. Storage: Stored at −65° C. ±15° C., material was thawed at room temperature, or overnight at 4° C. Thawed protein was stored at 4° C. for no longer than 7 days. 2 mg/ml protein solution administered at 5 ml/kg by i.p. injection.

Cisplatin injection (APP Pharmaceuticals LLC, Schaumburg, Il., USA). Product No 100365, Lot No 6103635. Storage: at room temperature protected from light. 1 mg/ml solution was administered at 5 ml/kg by i.v. injection.

### Experimental Procedures

Nasal Septum Carcinoma cells (RPMI2650, ATCC# CCL-30) were injected subcutaneously in the right flank (5×10E6 in 0.1 ml of SFM) of 8-week old female mice. Nine days following implantation animals were randomized into 4 groups based on tumor volume. Animal dosing and tumor measurement was done three times a week.

TABLE 4
Study cohorts

| Group | N | Treatment | Dose | Route | Schedule |
|---|---|---|---|---|---|
| 1 | 12 | mTBS | Volume | i.p. | Tiw ×3 |
| 2 | 10 | ACE-041 | 10 mg/kg | i.p. | Tiw ×3 |
| 3 | 10 | Cisplatin | 5 mg/kg | i.v. | qw ×3 |
| 4 | 10 | ACE-041 + | 10 mg/kg | i.p. and i.v. | Tiw + qw ×3 |
| | | Cisplatin | 5 mg/kg | | |

Tumor implantation. RPMI2650 (ATCC #CCL-30) were grown in Eagle's Minimum Essential Medium (ATCC Cat. No 30-2003), supplemented with fetal bovine serum (Gibco, Cat. No 26140) to a final concentration of 10%. Cell were harvested by mild trypsin digestion, washed in SFM and resuspended at a concentration of 50×10E6/ml in SFM. 0.1 ml (5×10E6 cells) of the cell suspension was injected subcutaneously in the right flank of a nude mouse.

Tumor measurement. Tumor size was measured manually using digital calipers. Tumor volume was calculated using the formula for a modified ellipse ((Length*Width2)/2).

Data Analysis. Graphing and statistical analysis of the tumor volume and body weight data was done using GraphPad Prism 5 software (GraphPad Software Inc., La Jolla, Calif.). Unpaired two-tailed t-test (with or w/o Welch correction) was used to determine statistical significance. TGI was calculated according to the formula:

TGI=(GMDV(C)−GMDV(T))*100/(GMDV(C)−1), where GMDV=Geomean(final volumes)/Geomean(intitial volumes)

### Results

Tumor growth data. Treatment with ACE-041 showed modest anti-tumor activity (32% TGI) in RPMI2650 xenograft model. Cisplatin, given at maximum tolerated dose, also showed modest tumor growth inhibition (35% TGI). Dalantercept combined with cisplatin showed greater inhibition of the tumor growth than either agent alone (59% TGI, p=0.0077 vs. cisplatin, p=0.0002 vs. ACE-041 monotherapy).

TABLE 5
Tumor volume data (geo mean, mm3)

| Study cohort | Day 9 | Day 30 |
|---|---|---|
| Vehicle | 188.3 | 1469 |
| ACE-041 | 181.4 | 1024 |
| Cisplatin | 185.8 | 1001 |
| ACE-041 + Cisplatin | 187.2 | 704.7 |

TABLE 6
Tumor growth inhibition calculation, day 30

| Study cohort | GMDV | TGI (%) |
|---|---|---|
| Vehicle | 7.8 | n/a |
| ACE-041 | 5.6 | 32 |
| Cisplatin | 5.4 | 35 |
| ACE-041 + Cisplatin | 3.8 | 59 |

TABLE 7
p-values, day 30

| | Treatment vs. | Combo vs. | Combo vs. |
|---|---|---|---|
| Study cohort | vehicle | cisplatin | ACE-041 |
| Vehicle | | | |
| ACE-041 | 0.0005 | | |
| Cisplatin | 0.0013 | | |
| ACE-041 + Cisplatin | <0.0001 | 0.0077 | 0.0002 |

Animal body weight data. Treatment with ACE-041 did not show any sign of acute toxicity as monitored by changes in animal body weight. In contrast, treatment with cisplatin resulted in significant body weight loss during the dosing period. Animals were not able to fully recover in between the doses causing cumulative body weight loss of ˜13% on average on day 30. Some animals lost as much as 20% body weight, triggering termination of the study per IACUC regulations. Cisplatin-mediated body weight loss was also apparent in the combination treatment group; interestingly, the 3^{rd} dose of cisplatin seemed to be better tolerated by animals in that group compared to cisplatin monotherapy group (although the difference in body weight did not reach statistical significance).

TABLE 8
Average changes in body weight, day 30

| Study cohort | Average, % |
|---|---|
| Vehicle | 6.2 |
| ACE-041 | 9.1 |
| Cisplatin | −12.9 |
| ACE-041 + Cisplatin | −7.4 |

### Conclusions

ACE-041 dosed at 10 mg/kg i.p. three times a week showed modest anti-tumor activity in RPMI2650 H&N xenograft model with 32% TGI after 3 weeks of dosing. There was a trend towards an increase in body weight in ACE-041 group compared to the control group, however, the difference was not statistically significant.

Cisplatin dosed at 5 mg/kg i.v. on a once weekly schedule also showed moderate tumor growth inhibition of 35% after 3 weeks of dosing. Treatment with cisplatin resulted in significant body weight loss during the dosing period. Animals were not able to fully recover after each dose causing cumulative body weight loss of ˜13% on average on day 30.

Combination treatment of ACE-041 with cisplatin showed additive anti-tumor effect: 59% TGI on day 30, which was significantly better than either cisplatin (p=0.0077) or ACE-041 (p=0.0002) monotherapy. Cisplatin-mediated body weight loss was also apparent in the combination treatment group. The 3rd dose of cisplatin seemed to be better tolerated by animals in that group compared to cisplatin monotherapy group (although the difference in body weight did not reach statistical significance). This data suggests that combination of dalantercept with cisplatin is beneficial to patients with squamous carcinoma of head and neck.

### Example 2

### Phase 2 Study of Dalantercept in Recurrent or Metastatic Squamous Cell Carcinoma of the Head and Neck

Limited treatments exist for patients with recurrent or metastatic squamous cell carcinoma of the head and neck (SCCHN) after platinum therapy and the prognosis remains poor. Activin receptor-like kinase 1 (ALK1) is, a member of the TGF-β superfamily, and is selectively expressed on activated endothelial cells and is involved in blood vessel maturation. ALK1 binds to ligands bone morphogenetic protein (BMP) 9 and 10 and results in phosphorylation of Smad 1/5/8. Dalantercept is an ALK1 receptor fusion protein and acts as a ligand trap to BMP 9 and 10. In a completed Phase 1 study, Dalantercept demonstrated activity in a subset of patients with SCCHN . This current Phase 2 study sought to determine the activity of single agent Dalantercept in patients with advanced or metastatic SCCHN.

Methods: 46 patients were enrolled and received Dalantercept SC Q3W at 80 mg (n=2), 0.6 mg/kg (n=13), and 1.2 mg/kg (n=31) until disease progression or unacceptable toxicity. Archived and optional biopsies and serum were collected for PD (pharmacodynamic) studies . The primary endpoint was response rate (RR) per RECIST 1.1 and secondary endpoints included progression free survival and overall survival. Key eligibility: recurrent or metastatic SCCHN of mucosal origin, >1 one prior platinum regimen, ECOG performance status ≦1, and no prior anti-angiogenic therapy. Results: 41 patients were evaluable (1 at 80 mg, 13 at 0.6 mg/kg, 27 at 1.2 mg/kg). The median age was 60.5 years, 85% M/15% F, ECOG: 0 (35%)/1(65%), HPV 41% positive/33% negative/26% unknown. Median # of prior therapies=4 and 61% patients had prior cetuximab. 1 patient at 1.2 mg/kg (2.4%) achieved a partial response (PR). The proportion of patients with stable disease (SD)>3 cycles was 23% (n=3) at 0.6 mg/kg and 37% at 1.2 mg/kg (n=10). Of those patients, with SD or better (n=16), 62% were known to be HPV+. The most common drug-related adverse events (AEs) were grade 1-2 and consisted of anemia, fatigue, peripheral edema, headache, hyponatremia, and pleural effusion. The frequency of grade >3 related AEs was 13% and the most common were hyponatremia (n=3) and pleural effusion (n=2).

### Conclusion

Dalantercept is an anti-angiogenic agent that inhibits ALK1 signaling and disrupts the process of blood vessel maturation. In this heavily pre-treated SCCHN population, Dalantercept demonstrated dose dependent, monotherapy activity and an overall acceptable safety profile.

### REFERENCES

All publications, patents, patent applications, publication, and database entries (e.g., sequence database entries) mentioned herein, e.g., in the Background, Summary, Detailed Description, Examples, and/or References sections, are hereby incorporated by reference in their entirety as if each individual publication, patent, patent application, publication, and database entry was specifically and individually incorporated herein by reference. In case of conflict, the present application, including any definitions herein, will control.

### SEQUENCES

```
SEQ ID NO: 1 - amino acid sequence of Human Activin receptor-like
kinase 1 (ALK-1, gi:3915750)
    1 MTLGSPRKGL LMLLMALVTQ GDPVKPSRGP LVTCTCESPH CKGPTCRGAW CTVVLVREEG
  61 RHPQEHRGCG NLHRELCRGR PTEFVNHYCC DSHLCNHNVS LVLEATQPPS EQPGTDGQLA
121 LILGPVLALL ALVALGVLGL WHVRRRQEKQ RGLHSELGES SLILKASEQG DSMLGDLLDS
181 DCTTGSGSGL PFLVQRTVAR QVALVECVGK GRYGEVWRGL WHGESVAVKI FSSRDEQSWF
241 RETEIYNTVL LRHDNILGFI ASDMTSRNSS TQLWLITHYH EHGSLYDFLQ RQTLEPHLAL
301 RLAVSAACGL AHLHVEIFGT QGKPAIAHRD FKSRNVLVKS NLQCCIADLG LAVMHSQGSD
361 YLDIGNNPRV GTKRYMAPEV LDEQIRTDCF ESYKWTDIWA FGLVLWEIAR RTIVNGIVED
421 YRPPFYDVVP NDPSFEDMKK VVCVDQQTPT IPNRLAADPV LSGLAQMMRE CWYPNPSARL
481 TALRIKKTLQ KISNSPEKPK VIQ
```

Single underlining shows the extracellular domain. Double underlining shows the intracellular domain. The signal peptide and the transmembrane domain are not underlined.

```
SEQ ID NO: 2 - nucleic acid sequence of Human Activin receptor-like kinase
1 (ALK-1)
      1 atgaccttgg gctcccccag gaaaggcctt ctgatgctgc tgatggcctt ggtgacccag
    61 ggagaccctg tgaagccgtc tcggggcccg ctggtgacct gcacgtgtga gagcccacat
  121 tgcaaggggc ctacctgccg gggggcctgg tgcacagtag tgctggtgcg ggaggagggg
  181 aggcaccccc aggaacatcg gggctgcggg aacttgcaca gggagctctg cagggggcgc
  241 cccaccgagt tcgtcaacca ctactgctgc gacagccacc tctgcaacca caacgtgtcc
  301 ctggtgctgg aggccaccca acctccttcg gagcagccgg gaacagatgg ccagctggcc
  361 ctgatcctgg gccccgtgct ggccttgctg gccctggtgg ccctgggtgt cctgggcctg
  421 tggcatgtcc gacggaggca ggagaagcag cgtggcctgc acagcgagct gggagagtcc
  481 agtctcatcc tgaaagcatc tgagcagggc gacagcatgt tgggggacct cctggacagt
  541 gactgcacca cagggagtgg ctcagggctc cccttcctgg tgcagaggac agtggcacgg
  601 caggttgcct tggtggagtg tgtgggaaaa ggccgctatg gcgaagtgtg gcggggcttg
  661 tggcacggtg agagtgtggc cgtcaagatc ttctcctcga gggatgaaca gtcctggttc
  721 cgggagactg agatctataa cacagtgttg ctcagacacg acaacatcct aggcttcatc
  781 gcctcagaca tgacctcccg caactcgagc acgcagctgt ggctcatcac gcactaccac
  841 gagcacggct ccctctacga ctttctgcag agacagacgc tggagcccca tctggctctg
  901 aggctagctg tgtccgcggc atgcggcctg gcgcacctgc acgtggagat cttcggtaca
  961 cagggcaaac cagccattgc ccaccgcgac ttcaagagcc gcaatgtgct ggtcaagagc
1021 aacctgcagt gttgcatcgc cgacctgggc ctggctgtga tgcactcaca gggcagcgat
1081 tacctggaca tcggcaacaa cccgagagtg ggcaccaagc ggtacatggc acccgaggtg
1141 ctggacgagc agatccgcac ggactgcttt gagtcctaca agtggactga catctgggcc
1201 tttggcctgg tgctgtggga gattgcccgc cggaccatcg tgaatggcat cgtggaggac
1261 tatagaccac ccttctatga tgtggtgccc aatgacccca gctttgagga catgaagaag
1321 gtggtgtgtg tggatcagca gacccccacc atccctaacc ggctggctgc agacccggtc
1381 ctctcaggcc tagctcagat gatgcgggag tgctggtacc caaacccctc tgcccgactc
1441 accgcgctgc ggatcaagaa gacactacaa aaaattagca acagtccaga gaagcctaaa
1501 gtgattcaat ag
```

The coding sequence is underlined. The portion encoding the extracellular domain is double underlined.

SEQ ID NO: 3 - amino acid sequence of an hALK1-
Fc Fusion Protein.
DPVKPSRGPLVTCTCESPHCKGPTCRGAWCTVVLVREEGRHPQEHRGCGN
LHRELCRGRPTEFVNHYCCDSHLCNHNVSLVLEATQPPSEQPGTDGQLAT
GGGTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKEY
KCKVSNKALPVPIEKTISKAKGQPREPQVYTLPPSREEMTKNQVSLTCLV
KGFYPSDIAVEWESNGQPENNYKTTPPVLDSDGSFFLYSKLTVDKSRWQQ
GNVFSCSVMHEALHNHYTQKSLSLSPGK

The hALK1-Fc protein includes amino acids 22-120 of the human ALK1 protein, fused at the C-terminus to a linker (underlined) and an IgG1 Fc region.

SEQ ID NO: 4 - amino acid sequence of an exemplary
ALK1-Fc fusion protein
DPVKPSRGPLVTCTCESPHCKGPTCRGAWCTVVLVREEGRHPQEERGCGN
LHRELCRGRPTEFVNHYCCDSHLCNHNVSLVLEATQPPSEQPGTDGQLAT
GGGTHTCPPCPAPEALGAPSVFLFPPKPKDTLMISRTPEVTCVVVDVSHE
DPEVKFNWYVDGVEVHNAKTKPREEQYNSTYRVVSVLTVLHQDWLNGKEY
KCKVSNKALPVPIEKTISKAKGQPREPQVYTLPPSREEMTKNQVSLTCLV
KGFYPSDIAVEWESNGQPENNYKTTPPVLDSDGPFFLYSKLTVDKSRWQQ
GNVFSCSVMHEALHNHYTQKSLSLSPGK

SEQ ID NO: 5 - an exemplary amino acid sequence of
an Fc domain
THTCPPCPAPELLGGPSVFLFPPKPKDTLMISRTPEVTCVVVD(A)VSHE
DPEVKFNWYVDGVEVHNAKTKPREEQYNSTYRVVSVLTVLHQDWLNGKEY
KCK(A)VSNKALPVPIEKTISKAKGQPREPQVYTLPPSREEMTKNQVSLT
CLVKGFYPSDIAVEWESNGQPENNYKTTPPVLDSDGPFFLYSKLTVDKSR
WQQGNVFSCSVMHEALHN(A)HYTQKSLSLSPGK*

In some embodiments, one or more of the underlined residues may be mutated, e.g., to Alanine (A), in order to modulate binding to the Fc receptor relative to a wild type Fc domain.

### EQUIVALENTS AND SCOPE

Those skilled in the art will recognize, or be able to ascertain using no more than routine experimentation, many equivalents of the embodiments described herein. The scope of the present disclosure is not intended to be limited to the above description, but rather is as set forth in the appended claims.

Articles such as “a,” “an,” and “the” may mean one or more than one unless indicated to the contrary or otherwise evident from the context. Claims or descriptions that include “or” between two or more members of a group are considered satisfied if one, more than one, or all of the group members are present, unless indicated to the contrary or otherwise evident from the context. The disclosure of a group that includes “or” between two or more group members provides embodiments in which exactly one member of the group is present, embodiments in which more than one members of the group are present, and embodiments in which all of the group members are present. For purposes of brevity those embodiments have not been individually spelled out herein, but it will be understood that each of these embodiments is provided herein and may be specifically claimed or disclaimed.

It is to be understood that this disclosure encompasses all variations, combinations, and permutations in which one or more limitation, element, clause, or descriptive term, from one or more of the claims or from one or more relevant portion of the description, is introduced into another claim. For example, a claim that is dependent on another claim can be modified to include one or more of the limitations found in any other claim that is dependent on the same base claim. Furthermore, where the claims recite a composition, it is to be understood that methods of making or using the composition according to any of the methods of making or using disclosed herein or according to methods known in the art, if any, are included, unless otherwise indicated or unless it would be evident to one of ordinary skill in the art that a contradiction or inconsistency would arise.

Where elements are presented as lists, e.g., in Markush group format, it is to be understood that every possible subgroup of the elements is also disclosed, and that any element or subgroup of elements can be removed from the group. It is also noted that the term “comprising” is intended to be open and permits the inclusion of additional elements or steps. It should be understood that, in general, where an embodiment, product, or method is referred to as comprising particular elements, features, or steps, embodiments, products, or methods that consist, or consist essentially of, such elements, features, or steps, are provided as well. For purposes of brevity those embodiments have not been individually spelled out herein, but it will be understood that each of these embodiments is provided herein and may be specifically claimed or disclaimed.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in some embodiments, to the tenth of the unit of the lower limit of the range, unless the context clearly dictates otherwise. For purposes of brevity, the values in each range have not been individually spelled out herein, but it will be understood that each of these values is provided herein and may be specifically claimed or disclaimed. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

In addition, it is to be understood that any particular embodiment disclosed herein may be explicitly excluded from any one or more of the claims. Where ranges are given, any value within the range may explicitly be excluded from any one or more of the claims. Any embodiment, element, feature, application, or aspect of the compositions and/or methods disclosed herein, can be excluded from any one or more claims. For purposes of brevity, all of the embodiments in which one or more elements, features, purposes, or aspects is excluded are not set forth explicitly herein.

1. A method of treating head and neck cancer in a subject, the method comprising administering to a subject in need thereof
(a) an agent selected from the group consisting of
(i) an ALK1-extracellular domain (ALK1-ECD) polypeptide;
(ii) an antibody that binds to an ALK1 polypeptide comprising amino acids 22-118 of SEQ ID NO: 1; or
(iii) an antibody that binds to BMP9 or BMP10; and
(b) a chemotherapeutic platinum agent;
in an amount sufficient to treat the head and neck cancer in the subject.

2. The method of claim 1, wherein the amino acid sequence of the ALK1-ECD polypeptide is at least 90% identical to the sequence of amino acids 22-118 of SEQ ID NO: 1.

3-4. (canceled)

5. The method of claim 1, wherein the C-terminal amino acid residue of the ALK1-ECD polypeptide is not a glutamine residue.

6. The method of claim 1, wherein the C-terminal amino acid residue of the ALK1-ECD polypeptide is proline 113 (P113), glycine 114 (G114), threonine 115 (T115), aspartic acid 116 (D116), glycine 117 (G117), leucine 119 (L119), alanine 120 (A120), leucine 121 (L121), isoleucine 122 (I122), or leucine 123 (L123) of SEQ ID NO: 1.

7. The method of claim 1, wherein the ALK1-ECD polypeptide is fused to an Fc portion of an immunoglobulin thus forming an ALK1-Fc fusion protein.

8-11. (canceled)

12. The method of claim 7, wherein at least 90% of the ALK1-Fc fusion protein is in a dimeric form.

14. The method of claim 1, wherein the ALK1-ECD polypeptide and/or the ALK1-Fc fusion protein binds BMP9 or BMP10 with a K_{D} of less than 1×10⁻⁷M.

15. The method of claim 1, wherein the ALK1-ECD polypeptide and/or the ALK1-Fc fusion protein binds to TGFβ-1 with a K_{D} of greater than 1×10⁻⁶M.

16-22. (canceled)

23. The method of claim 1, wherein the chemotherapeutic platinum agent comprises a coordination complex of platinum.

24. The method of claim 1, wherein the chemotherapeutic platinum agent is cisplatin, carboplatin, oxaliplatin, satraplatin, picoplatin, Nedaplatin, Triplatin, Lipoplatin, or any combination thereof.

25. The method of claim 1, wherein the ALK1-ECD polypeptide and/or the ALK1-Fc fusion protein is administered to the subject at a dosage of 0.1-30 mg/kg/day; and/or wherein the chemotherapeutic platinum agent is administered to the subject at a dosage of 0.1, 0.2, 0.3, 0.4, 0.5, 0.6, 0.7, 0.8, 0.9, 1, 1.5, 2, 2.5, 3, 3.5, 4, 4.5, 5, 6, 7, 7.5, 8, 9, or 10 mg/kg/day.

26-28. (canceled)

29. The method of claim 1, wherein the ALK1-ECD polypeptide and/or the ALK1-Fc fusion protein is administered to the subject at a dose of 10 mg/kg/day, and the chemotherapeutic platinum agent is administered to the subject at a dose of 5 mg/kg/day.

30. The method of claim 1, wherein the head and neck cancer is positive for human papilloma virus (HPV).

31-32. (canceled)

33. The method of claim 1, wherein the head and neck cancer is a recurrent or metastatic squamous cell carcinoma.

34. A method of treating HPV-positive head and neck cancer in a subject, the method comprising administering to a subject in need thereof an agent selected from the group consisting of
(i) an ALK1-extracellular domain (ALK1-ECD) polypeptide;
(ii) an antibody that binds to an ALK1 polypeptide comprising amino acids 22-118 of SEQ ID NO: 1; and
(iii) an antibody that binds to BMP9 or BMP10;
in an amount sufficient to treat the head and neck cancer in the subject.

35-61. (canceled)

62. The method of claim 34, wherein the method further comprises administering a chemotherapeutic platinum agent to the subject.

63-67. (canceled)

68. A method, comprising:
(a) determining whether a head and neck cancer in a subject is human papilloma virus (HPV)-positive;
wherein, if the head and neck cancer is positive for HPV, then the cancer is identified to respond to treatment with an agent selected from the group consisting of
(i) an ALK1-extracellular domain (ALK1-ECD) polypeptide;
(ii) an antibody that binds to an ALK1 polypeptide comprising amino acids 22-118 of SEQ ID NO: 1; and
(iii) an antibody that binds to BMP9 or BMP10; and
(b) administering the agent to the subject in an amount effective to treat the head and neck cancer.

69-84. (canceled)

85. A method, comprising:
(a) obtaining a biopsy from a head and neck cancer in a subject;
(b) determining whether the head and neck cancer is HPV positive; and
(c) if the head and neck cancer is positive for HPV, identifying the head and neck cancer as responsive to treatment with an agent selected from the group consisting of
(i) an ALK1-extracellular domain (ALK1-ECD) polypeptide;
(ii) an antibody that binds to an ALK1 polypeptide comprising amino acids 22-118 of SEQ ID NO: 1; and
(iii) an antibody that binds to BMP9 or BMP10.

86. The method of claim 85, wherein the method further comprises administering the agent to the subject in an amount effective to treat the head and neck cancer.

87-101. (canceled)

102. A pharmaceutical composition for the treatment of head and neck cancer in a subject, the composition comprising:
(a) an agent selected from the group consisting of
(i) an ALK1-extracellular domain (ALK1-ECD) polypeptide;
(ii) an antibody that binds to BMP9 or BMP10; or
(iii) an antibody that binds to an ALK1 polypeptide comprising amino acids 22-118 of SEQ ID NO: 1; and
(b) a chemotherapeutic platinum agent,
wherein the agent of (a) and the chemotherapeutic platinum agent are in an amount sufficient to treat a head and neck cancer in the subject.

103-124. (canceled)


SEQUENCE LISTING (5 sequences)

SEQ ID NO: 1 - protein, 503 aa, Homo sapiens
MTLGSPRKGLLMLLMALVTQGDPVKPSRGPLVTCTCESPHCKGPTCRGAWCTVVLVREEGRHPQEHRGCGNLHRELCRGRPTEFVNHYCCDSHLCNHNVSLVLEATQPPSEQPGTDGQLALILGPVLALLALVALGVLGLWHVRRRQEKQRGLHSELGESSLILKASEQGDSMLGDLLDSDCTTGSGSGLPFLVQRTVARQVALVECVGKGRYGEVWRGLWHGESVAVKIFSSRDEQSWFRETEIYNTVLLRHDNILGFIASDMTSRNSSTQLWLITHYHEHGSLYDFLQRQTLEPHLALRLAVSAACGLAHLHVEIFGTQGKPAIAHRDFKSRNVLVKSNLQCCIADLGLAVMHSQGSDYLDIGNNPRVGTKRYMAPEVLDEQIRTDCFESYKWTDIWAFGLVLWEIARRTIVNGIVEDYRPPFYDVVPNDPSFEDMKKVVCVDQQTPTIPNRLAADPVLSGLAQMMRECWYPNPSARLTALRIKKTLQKISNSPEKPKVIQ

SEQ ID NO: 2 - DNA, 1512 bp, Homo sapiens
atgaccttgggctcccccaggaaaggccttctgatgctgctgatggccttggtgacccagggagaccctgtgaagccgtctcggggcccgctggtgacctgcacgtgtgagagcccacattgcaaggggcctacctgccggggggcctggtgcacagtagtgctggtgcgggaggaggggaggcacccccaggaacatcggggctgcgggaacttgcacagggagctctgcagggggcgccccaccgagttcgtcaaccactactgctgcgacagccacctctgcaaccacaacgtgtccctggtgctggaggccacccaacctccttcggagcagccgggaacagatggccagctggccctgatcctgggccccgtgctggccttgctggccctggtggccctgggtgtcctgggcctgtggcatgtccgacggaggcaggagaagcagcgtggcctgcacagcgagctgggagagtccagtctcatcctgaaagcatctgagcagggcgacagcatgttgggggacctcctggacagtgactgcaccacagggagtggctcagggctccccttcctggtgcagaggacagtggcacggcaggttgccttggtggagtgtgtgggaaaaggccgctatggcgaagtgtggcggggcttgtggcacggtgagagtgtggccgtcaagatcttctcctcgagggatgaacagtcctggttccgggagactgagatctataacacagtgttgctcagacacgacaacatcctaggcttcatcgcctcagacatgacctcccgcaactcgagcacgcagctgtggctcatcacgcactaccacgagcacggctccctctacgactttctgcagagacagacgctggagccccatctggctctgaggctagctgtgtccgcggcatgcggcctggcgcacctgcacgtggagatcttcggtacacagggcaaaccagccattgcccaccgcgacttcaagagccgcaatgtgctggtcaagagcaacctgcagtgttgcatcgccgacctgggcctggctgtgatgcactcacagggcagcgattacctggacatcggcaacaacccgagagtgggcaccaagcggtacatggcacccgaggtgctggacgagcagatccgcacggactgctttgagtcctacaagtggactgacatctgggcctttggcctggtgctgtgggagattgcccgccggaccatcgtgaatggcatcgtggaggactatagaccacccttctatgatgtggtgcccaatgaccccagctttgaggacatgaagaaggtggtgtgtgtggatcagcagacccccaccatccctaaccggctggctgcagacccggtcctctcaggcctagctcagatgatgcgggagtgctggtacccaaacccctctgcccgactcaccgcgctgcggatcaagaagacactacaaaaaattagcaacagtccagagaagcctaaagtgattcaatag

SEQ ID NO: 3 - protein, 328 aa, Artificial Sequence
  Synthetic Polypeptide
DPVKPSRGPLVTCTCESPHCKGPTCRGAWCTVVLVREEGRHPQEHRGCGNLHRELCRGRPTEFVNHYCCDSHLCNHNVSLVLEATQPPSEQPGTDGQLATGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPVPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 328 aa, Artificial Sequence
  Synthetic Polypeptide
DPVKPSRGPLVTCTCESPHCKGPTCRGAWCTVVLVREEGRHPQEERGCGNLHRELCRGRPTEFVNHYCCDSHLCNHNVSLVLEATQPPSEQPGTDGQLATGGGTHTCPPCPAPEALGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPVPIEKTISKAKGQPREPQVYTLPPSREEMTKNQVSLTCLVKGFYPSDIAVEWESNGQPENNYKTTPPVLDSDGPFFLYSKLTVDKSRWQQGNVFSCSVMHEALHNHYTQKSLSLSPGK

SEQ ID NO: 5 - protein, 225 aa, Artificial Sequence
  Synthetic Polypeptide
THTCPPCPAPELLGGPSVFLFPPKPKDTLMISRTPEVTCVVVXVSHEDPEVKFNWYVDGVEVHNAKTKPREEQYNSTYRVVSVLTVLHQDWLNGKEYKCXVSNKALPVPIEKTISKAKGQPREPQVYTLPPSREEMTKNQVSLTCLVKGFYPSDIAVEWESNGQPENNYKTTPPVLDSDGPFFLYSKLTVDKSRWQQGNVFSCSVMHEALHXHYTQKSLSLSPGK